(19) United States
(12) Patent Application Publication
(10) Pub. No.: US 2009/0274674 A1
(43) Pub. Date: Nov. 5, 2009

(54) Heterocyclic Oxime Compounds, Process for Their Preparation and Pharmaceutical Compositions Containing Them

(75) Inventors: Franck Suzenet, Saint Jean de Bray (FR); Gerald Guillaumet, Saint Jean Le Blanc (FR); Christelle Pillard, La Celle Conde (FR); Carine Bassene, Saint Jean de Bray (FR); Catherine Dacquet, Paris (FR); Alain Ktorza, Nogent sur Marne (FR); Daniel-Henri Caignard, Boisemont (FR)

Correspondence Address: THE FIRM OF HUESCHEN AND SAGE, SEVENTH FLOOR, KALAMAZOO BUILDING, 107 WEST MICHIGAN AVENUE, KALAMAZOO, MI, 49007, US

(73) Assignee: LES LABORATOIRES SERVIER, Courbevoie Cedex (FR)

(21) Appl. No.: 12/086,785

(22) PCT Filed: Dec. 19, 2006
(86) PCT No.: PCT/FR2006/002778

§ 371 Date: Jun. 19, 2008

(30) Foreign Application Priority Data
Dec. 20, 2005 (FR) 0512924

(51) Int. Cl. A61K 31/122; C07D 471/04; A61K 31/437; A61P 3/04

(52) U.S. Cl. 424/94.1; 546/113; 514/300

(57) ABSTRACT

Compounds of formula (I):

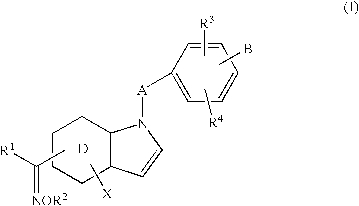

wherein:
X represents a hydrogen or halogen atom or an alkyl group,
R¹, R², R³ and R⁴ are as defined in the description,
A represents an alkylene chain as defined in the description,
B represents an alkyl or alkenyl group substituted by a group

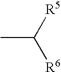

D represents a pyridine nucleus.

The present invention relates to new heterocyclic oxime compounds, to a process for their preparation and to pharmaceutical compositions containing them.

The compounds described in the present invention are new and have pharmacological properties that are of special interest: they are excellent hypoglycaemic and hypolipaemic agents.

The treatment of non-insulin-dependent type II diabetes remains unsatisfactory despite the introduction onto the market of a large number of oral hypoglycaemic compounds intended to facilitate the secretion of insulin and to promote its action in peripheral target tissues.

During the last ten years, a class of compounds having a thiazolidinedione structure (U.S. Pat. No. 5,089,514, U.S. Pat. No. 5,306,726) has demonstrated a marked anti-diabetic activity by promoting sensitivity to insulin in the target peripheral tissues (skeletal muscle, liver, adipose tissue) of animal models having non-insulin-dependent type II diabetes. Those compounds also lower the levels of insulin and levels of lipids in the same animal models and induce in vitro differentiation of preadipocyte cell lines into adipocyte cell lines (A. Hiragun et al., J. Cell. Physiol., 1988, 134, 124-130; R. F. Kleitzen et al., Mol. Pharmacol., 1992, 41, 393-398).

The treatment of preadipocyte cell lines with the thiazolidinedione rosiglitazone brings about induction of the expression of specific genes of the lipid metabolism, such as aP2 and adipsin, and also the expression of the glucose transporters GLUT1 and GLUT4, suggesting that the effect of the thiazolidinediones observed in vivo may be mediated via adipose tissue. That specific effect is obtained by the stimulation of nuclear transcription factors <<peroxisome proliferator-activated receptor gamma>> (PPAR γ2). Such compounds are capable of restoring sensitivity to insulin in peripheral tissues, such as adipose tissue or skeletal muscle (J. E. Gerich, New Engl. Med., 19, 321, 1231-1245).

Compounds having a thiazolidinedione structure (troglitazone, rosiglitazone) have demonstrated disturbing side effects in man, however, especially liver problems (Script N^{o} 2470, 1999, Sep. 8^{th}, 25).

A large number of hypoglycaemic agents have significant side effects (hepatic, cardiac, haematopoietic), which limit their long-term use in the treatment of non-insulin-dependent type II diabetes.

The development of new therapeutic agents that are less toxic and that are active over the long term is absolutely necessary in this pathology.

Moreover, hyperlipidaemia is often observed in diabetics (Diabetes Care, 1995, 18 (supplement 1), 86/8/93). The association of hyperglycaemia with hyperlipidaemia increases the risk of cardiovascular disease in diabetics. Hyperglycaemia, hyperlipidaemia and obesity have become pathologies of the modern world marked by the intake of food in large quantities and a chronic lack of exercise.

The increase in frequency of those pathologies calls for the development of new therapeutic agents that are active in such disorders: compounds having an excellent hypoglycaemic and hypolipaemic activity whilst avoiding the side effects observed with thiazolidinediones are consequently very beneficial in the treatment and/or prophylaxis of those pathologies, and are indicated especially in the treatment of non-insulin-dependent type II diabetes for reducing peripheral insulin resistance and for normalising glucose control.

In addition to the fact that they are new, the compounds of the present invention meet the above pharmacological criteria and are excellent hypoglycaemic and hypolipaemic agents.

The present invention relates more especially to compounds of formula (I):

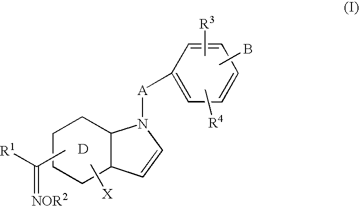

wherein:
R¹ represents an aryl, heteroaryl or (C₃-C₈)cycloalkyl group,
R² represents a hydrogen atom, or a linear or branched (C₁-C₆)alkyl group, an aryl group or an aryl-(C₁-C₆)alkyl group in which the alkyl moiety may be linear or branched,
X represents a hydrogen or halogen atom or a linear or branched (C₁-C₆)alkyl group,
R³ and R⁴, which may be identical or different, each represent a hydrogen or halogen atom, a linear or branched (C₁-C₆)alkyl, linear or branched (C₁-C₆)alkoxy or linear or branched (C₁-C₆)alkylamino group or a di(C₁-C₆)alkylamino group in which the alkyl moieties may be linear or branched,
D represents a pyridine nucleus,
A represents a (C₁-C₆)alkylene chain in which a CH₂ group may be replaced by a hetero atom selected from oxygen and sulphur, or by an NRₐ group (wherein Rₐ represents a hydrogen atom or a linear or branched (C₁-C₆)alkyl group),
B represents a linear or branched (C₁-C₆)alkyl group or a linear or branched (C₂-C₆)alkenyl group, those groups being substituted by a group of formula (II):

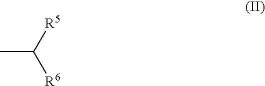

wherein R⁵ represents a —COOR group and R⁶ represents an —OR′ group wherein R and R′, which may be identical or different, each represent a hydrogen atom, or a linear or branched (C₁-C₆)alkyl group unsubstituted or substituted by one or more halogen atoms,
   it being understood that:
the oxime R¹—C(═N—OR²)— may have the Z or E configuration,
aryl is understood to mean a group: phenyl, naphthyl or biphenyl, wherein those groups may be partially hydrogenated,
heteroaryl is understood to mean any aromatic mono- or bi-cyclic group containing from 5 to 10 ring members, which in the case of bicyclic heteroaryl groups may be partially hydrogenated on one of the rings, and containing from 1 to 3 hetero atoms selected from oxygen, nitrogen and sulphur,
   wherein the aryl and heteroaryl groups so defined may be substituted by from 1 to 3 groups selected from linear or branched (C₁-C₆)alkyl, linear or branched (C₁-C₆)polyhaloalkyl, linear or branched (C₁-C₆)alkoxy, hydroxy, carboxy, formyl, NR_{b}R_{c} (wherein R_{b} and R_{c}, which may be identical or different, each represent a hydrogen atom, a linear or branched (C₁-C₆)alkyl group, an aryl group or a heteroaryl group), ester, amido, nitro, cyano, and halogen atoms,
   to their enantiomers and diastereoisomers, and also to pharmaceutically acceptable addition salts thereof with an acid or a base.

Amongst the pharmaceutically acceptable acids there may be mentioned, without implying any limitation, hydrochloric acid, hydrobromic acid, sulphuric acid, phosphonic acid, acetic acid, trifluoroacetic acid, lactic acid, pyruvic acid, malonic acid, succinic acid, glutaric acid, fumaric acid, tartaric acid, maleic acid, citric acid, ascorbic acid, methanesulphonic acid, camphoric acid, oxalic acid, etc. . . .

Amongst the pharmaceutically acceptable bases there may be mentioned, without implying any limitation, sodium hydroxide, potassium hydroxide, triethylamine, tert-butylamine, etc.

Preferred compounds of the invention are compounds of formula (I) wherein R¹ represents an aryl group and more especially a phenyl group.

A preferred R² group of the compounds of formula (I) according to the invention is the alkyl group and more especially the methyl group.

More especially, the compounds of formula (I) according to the invention are compounds wherein D with the ring to which it is fused represent a 1H-pyrrolo[2,3-b]pyridine system.

A preferred group for R³ and R⁴ is the hydrogen atom.

Preferably, A represents an alkylene chain in which a CH₂ group is replaced by an oxygen atom.

More especially, the invention relates to compounds of formula (I) wherein A represents an ethyleneoxy group.

X preferably represents a hydrogen atom.

The preferred R⁵ group is the COOH or COOMe group.

The preferred R⁶ group is the OEt or OCH₂CF₃ group.

The preferred B group is the group —CH₂—CH(R⁵)(R⁶) and more especially the group —CH₂—CH(R⁵)(R⁶) wherein R⁵ represents a COOH, COOMe or COOEt group and R⁶ represents a methoxy, ethoxy or trifluoroethoxy group.

Very advantageously, the invention relates to compounds of formula (I) having a 1H-pyrrolo[2,3-b]pyridine structure wherein:

X represents a hydrogen atom,
R¹ represents a phenyl, cyclopropyl, cyclopentyl or cyclohexyl group,
A represents a —CH₂—CH₂—O— chain,
R represents a methyl group,
R³ and R⁴ simultaneously represent a hydrogen atom,
B represents a —CH₂—CH(R⁵)(R⁶) group wherein R⁵ represents a COOMe, COOEt or COOH group and R⁶ represents an ethoxy or trifluoroethoxy group.

Even more especially, the invention relates to the following compounds of formula (I):
methyl 2-ethoxy-3-[4-(2-{6-[(E)-(methoxyimino)(phenyl)methyl]-1H-pyrrolo[2,3-b]pyridin-1-yl}ethoxy)phenyl]propanoate,
methyl (2S)-2-ethoxy-3-[4-(2-{6-[(E)-(methoxyimino)(phenyl)methyl]-1H-pyrrolo[2,3-b]pyridin-1-yl}ethoxy)phenyl]propanoate,
methyl 3-[4-(2-{6-[(E)-(methoxyimino)(phenyl)methyl]-1H-pyrrolo[2,3-b]pyridin-1-yl}ethoxy)phenyl]-2-(2,2,2-trifluoroethoxy)propanoate,
methyl (2S)-3-[4-(2-{6-[(E)-(methoxyimino)(phenyl)methyl]-1H-pyrrolo[2,3-b]pyridin-1-yl}ethoxy)phenyl]-2-(2,2,2-trifluoroethoxy)propanoate,
2-ethoxy-3-[4-(2-{6-[(E)-(methoxyimino)(phenyl)methyl]-1H-pyrrolo[2,3-b]pyridin-1-yl}ethoxy)phenyl]propanoic acid,
(2S)-2-ethoxy-3-[4-(2-{6-[(E)-(methoxyimino)(phenyl)methyl]-1H-pyrrolo[2,3-b]pyridin-1-yl}ethoxy)phenyl]propanoic acid,
3-[4-(2-{6-[(E)-(methoxyimino)(phenyl)methyl]-1H-pyrrolo[2,3-b]pyridin-1-yl}-ethoxy)phenyl]-2-(2,2,2-trifluoroethoxy)propanoic acid,
(2S)-3-[4-(2-{6-[(E)-(methoxyimino)(phenyl)methyl]-1H-pyrrolo[2,3-b]pyridin-1-yl}-ethoxy)phenyl]-2-(2,2,2-trifluoroethoxy)propanoic acid,
methyl 2-ethoxy-3-[4-(2-{6-[(Z)-(methoxyimino)(phenyl)methyl]-1H-pyrrolo[2,3-b]pyridin-1-yl}ethoxy)phenyl]propanoate,
2-ethoxy-3-[4-(2-{6-[(Z)-(methoxyimino)(phenyl)methyl]-1H-pyrrolo[2,3-b]pyridin-1-yl}ethoxy)phenyl]propanoic acid,
methyl 2-ethoxy-3-[4-(2-{5-[(methoxyimino)(phenyl)methyl]-1H-pyrrolo[2,3-b]pyridin-1-yl}ethoxy)phenyl]propanoate,
methyl 3-[4-(2-{5-[(methoxyimino)(phenyl)methyl]-1H-pyrrolo[2,3-b]pyridin-1-yl}-ethoxy)phenyl]-2-(2,2,2-trifluoroethoxy)propanoate,
2-ethoxy-3-[4-(2-{5-[(methoxyimino)(phenyl)methyl]-1H-pyrrolo[2,3-b]pyridin-1-yl}ethoxy)phenyl]propanoic acid,
3-[4-(2-{5-[(methoxyimino)(phenyl)methyl]-1H-pyrrolo[2,3-b]pyridin-1-yl}ethoxy)-phenyl]-2-(2,2,2-trifluoroethoxy)propanoic acid,
methyl 3-[4-(2-{4-chloro-5-[(Z)-(methoxyimino)(phenyl)methyl]-1H-pyrrolo[2,3-b]pyridin-1-yl}ethoxy)phenyl]-2-(2,2,2-trifluoroethoxy)propanoate,
3-[4-(2-{4-chloro-5-[(Z)-(methoxyimino)(phenyl)methyl]-1H-pyrrolo[2,3-b]pyridin-1-yl}ethoxy)phenyl]-2-(2,2,2-trifluoroethoxy)propanoic acid,
methyl 3-[4-(2-{6-[cyclopropyl(methoxyimino)methyl]-1H-pyrrolo[2,3-b]pyridin-1-yl}ethoxy)phenyl]-2-ethoxypropanoate,
methyl 3-[4-(2-{6-[cyclopropyl(methoxyimino)methyl]-1H-pyrrolo[2,3-b]pyridin-1-yl}ethoxy)phenyl]-2-(2,2,2-trifluoroethoxy)propanoate,
methyl 3-[4-(2-{6-[cyclohexyl(methoxyimino)methyl]-1H-pyrrolo[2,3-b]pyridin-1-yl}ethoxy)phenyl]-2-ethoxypropanoate,
methyl (2S)-3-[4-(2-{6-[cyclohexyl(methoxyimino)methyl]-1H-pyrrolo[2,3-b]pyridin-1-yl}ethoxy)phenyl]-2-ethoxypropanoate,
methyl 3-[4-(2-{6-[cyclohexyl(methoxyimino)methyl]-1H-pyrrolo[2,3-b]pyridin-1-yl}ethoxy)phenyl]-2-(2,2,2-trifluoroethoxy)propanoate,
methyl (2S)-3-[4-(2-{6-[cyclohexyl(methoxyimino)methyl]-1H-pyrrolo[2,3-b]pyridin-1-yl}ethoxy)phenyl]-2-(2,2,2-trifluoroethoxy)propanoate,
3-[4-(2-{6-[cyclopropyl(methoxyimino)methyl]-1H-pyrrolo[2,3-b]pyridin-1-yl}-ethoxy)phenyl]-2-ethoxypropanoic acid,
3-[4-(2-{6-[cyclopropyl(methoxyimino)methyl]-1H-pyrrolo[2,3-b]pyridin-1-yl}-ethoxy)phenyl]-2-(2,2,2-trifluoroethoxy)propanoic acid,
3-[4-(2-{6-[cyclohexyl(methoxyimino)methyl]-1H-pyrrolo[2,3-b]pyridin-1-yl}ethoxy)-phenyl]-2-ethoxypropanoic acid,
(2S)-3-[4-(2-{6-[cyclohexyl(methoxyimino)methyl]-1H-pyrrolo[2,3-b]pyridin-1-yl}-ethoxy)phenyl]-2-ethoxypropanoic acid,
3-[4-(2-{6-[cyclohexyl(methoxyimino)methyl]-1H-pyrrolo[2,3-b]pyridin-1-yl}ethoxy)-phenyl]-2-(2,2,2-trifluoroethoxy)propanoic acid,
(2S)-3-[4-(2-{6-[cyclohexyl(methoxyimino)methyl]-1H-pyrrolo[2,3-b]pyridin-1-yl}-ethoxy)phenyl]-2-(2,2,2-trifluoroethoxy)propanoic acid,
methyl 2-ethoxy-3-[4-(2-{6-[(methoxyimino)(4-pyridinyl)methyl]-1H-pyrrolo[2,3-b]pyridin-1-yl}ethoxy)phenyl]propanoate,
2-ethoxy-3-[4-(2-{6-[(methoxyimino) (4-pyridinyl)methyl]-1H-pyrrolo[2,3-b]pyridin-1-yl}ethoxy)phenyl]propanoic acid,
methyl (2S)-3-[4-(2-{6-[(methoxyimino)(phenyl)methyl]-1H-pyrrolo[2,3-b]pyridin-1-yl}ethoxy)phenyl]-2-(2,2,2-trifluoroethoxy)propanoate,
(2S)-3-[4-(2-{6-[(methoxyimino)(phenyl)methyl]-1H-pyrrolo[2,3-b]pyridin-1-yl}-ethoxy)phenyl]-2-(2,2,2-trifluoroethoxy)propanoic acid,
methyl 2-ethoxy-3-[4-(2-{6-[(phenoxyimino)(phenyl)methyl]-1H-pyrrolo[2,3-b]pyridin-1-yl}ethoxy)phenyl]propanoate,
2-ethoxy-3-[4-(2-{6-[(phenoxyimino)(phenyl)methyl]-1H-pyrrolo[2,3-b]pyridin-1-yl}ethoxy)phenyl]propanoic acid,
methyl 3-[4-(2-{6-[cyclopentyl(methoxyimino)methyl]-1H-pyrrolo[2,3-b]pyridin-1-yl}ethoxy)phenyl]-2-ethoxypropanoate,
3-[4-(2-{6-[cyclopentyl(methoxyimino)methyl]-1H-pyrrolo[2,3-b]pyridin-1-yl}ethoxy)-phenyl]-2-ethoxypropanoic acid,
methyl 2-ethoxy-3-[4-(2-{6-[(methoxyimino)(phenyl)methyl]-1H-pyrrolo[2,3-b]pyridin-1-yl}ethoxy)phenyl]propanoate,
2-ethoxy-3-[4-(2-{6-[(methoxyimino)(phenyl)methyl]-1H-pyrrolo[2,3-b]pyridin-1-yl}ethoxy)phenyl]propanoic acid,
3-[4-(2-{6-[cyclopropyl(hydroxyimino)methyl]-1H-pyrrolo[2,3-b]pyridin-1-yl}ethoxy)phenyl]-2-(2,2,2-trifluoroethoxy)propanoic acid,
2-ethoxy-3-[4-(2-{6-[(hydroxyimino)(phenyl)methyl]-1H-pyrrolo[2,3-b]pyridin-1-yl}ethoxy)phenyl]propanoic acid,
3-[4-(2-{6-[cyclopropyl(hydroxyimino)methyl]-1H-pyrrolo[2,3-b]pyridin-1-yl})ethoxy)phenyl]-2-ethoxypropanoic acid.

The enantiomers, diastereoisomers, and also pharmaceutically acceptable addition salts with an acid or a base of the preferred compounds of the invention form an integral part of the invention.

The present invention relates also to a process for the preparation of the compounds of formula (I) which is characterised in that there is used as starting material a compound of formula (III):

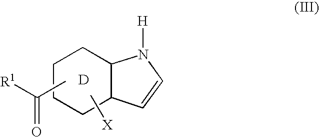

wherein D, R¹ and X are as defined for formula (I),
which is condensed in basic medium with a compound of formula (IV):

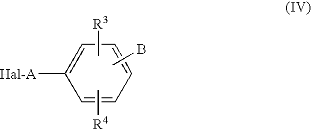

wherein A, B, R³ and R⁴ are as defined for formula (I) and Hal represents a halogen atom, to yield a compound of formula (V):

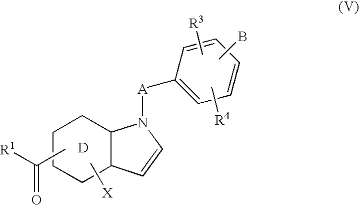

wherein R¹, R³, R⁴, A, B, D and X are as defined for formula (I),
which is subjected to the action of a compound of formula R²⁰—NH₂ wherein R² is as defined for formula (I) to yield a compound of formula (I):

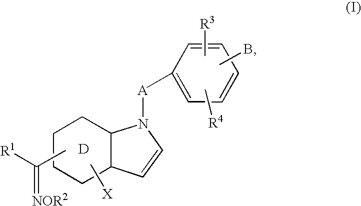

which may be purified according to a conventional separation technique, is converted, if desired, into addition salts with a pharmaceutically acceptable acid or base, and is optionally separated into isomers according to a conventional separation technique.

An advantageous variant relates to a process for the preparation of the compounds of formula (I) which is characterised in that there is used as starting material a compound of formula (III):

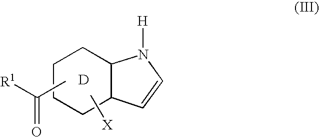

wherein D, R¹ and X are as defined for formula (I),
which is condensed with a compound of formula R²O—NH₂ wherein R² is as defined for formula (I) to yield a compound of formula (VI):

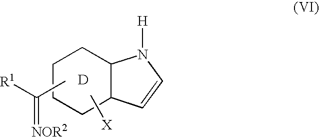

wherein R¹, R², D and X are as defined for formula (I),
which is condensed in basic medium with a compound of formula (IV):

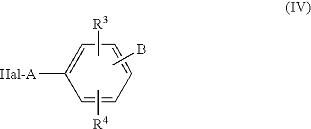

wherein A, B, R³ and R⁴ are as defined for formula (I) and Hal represents a halogen atom, to yield a compound of formula (I):

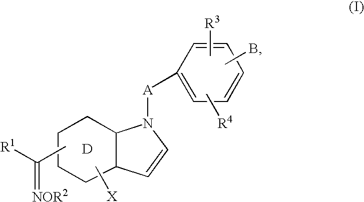

which may be purified according to a conventional separation technique, is converted, if desired, into addition salts with a pharmaceutically acceptable acid or base, and is optionally separated into isomers according to a conventional separation technique.

The compounds of formula (III) are commercial products or are readily obtainable by the person skilled in the art by conventional chemical reactions or are described in the literature.

The invention relates also to the compounds of formula (V):

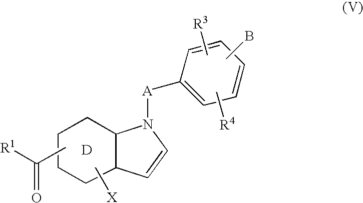

wherein D, X, A, R¹, R³, R⁴ and B are as defined for compounds of formula (I), for use as intermediates in the synthesis of the compounds of formula (I) and as hypoglycaemic and hypolipaemic agents.

Preferred compounds of formula (V) according to the invention are those wherein R¹ represents a (C₃-C₈)cycloalkyl group.

Even more especially, preferred compounds of formula (V) are:
methyl 3-(4-{2-[6-(cyclopropylcarbonyl)-1H-pyrrolo[2,3-b]pyridin-1-yl]ethoxy}-phenyl)-2-ethoxypropanoate,
methyl 3-(4-{2-[6-(cyclopropylcarbonyl)-1H-pyrrolo[2,3-b]pyridin-1-yl]ethoxy}-phenyl)-2-(2,2,2-trifluoroethoxy)propanoate,
methyl 3-(4-{2-[6-(cyclohexylcarbonyl)-1H-pyrrolo[2,3-b]pyridin-1-yl]ethoxy}-phenyl)-2-ethoxypropanoate,
methyl (2S)-3-(4-{2-[6-(cyclohexylcarbonyl)-1H-pyrrolo[2,3-b]pyridin-1-yl]ethoxy}-phenyl)-2-ethoxypropanoate,
methyl 3-(4-{2-[6-(cyclohexylcarbonyl)-1H-pyrrolo[2,3-b]pyridin-1-yl]ethoxy}-phenyl)-2-(2,2,2-trifluoroethoxy)propanoate,
methyl (2S)-3-(4-{2-[6-(cyclohexylcarbonyl)-1H-pyrrolo[2,3-b]pyridin-1-yl]ethoxy}-phenyl)-2-(2,2,2-trifluoroethoxy)propanoate,
3-(4-{2-[6-(cyclopropylcarbonyl)-1H-pyrrolo[2,3-b]pyridin-1-yl]ethoxy}phenyl)-2-ethoxypropanoic acid,
3-(4-{2-[6-(cyclopropylcarbonyl)-1H-pyrrolo[2,3-b]pyridin-1-yl]ethoxy}phenyl)-2-(2,2,2-trifluoroethoxy)propanoic acid,
3-(4-{2-[6-(cyclohexylcarbonyl)-1H-pyrrolo[2,3-b]pyridin-1-yl]ethoxy}phenyl)-2-ethoxypropanoic acid,
(2S)-3-(4-{2-[6-(cyclohexylcarbonyl)-1H-pyrrolo[2,3-b]pyridin-1-yl]ethoxy}phenyl)-2-ethoxypropanoic acid,
3-(4-{2-[6-(cyclohexylcarbonyl)-1H-pyrrolo[2,3-b]pyridin-1-yl]ethoxy}phenyl)-2-(2,2,2-trifluoroethoxy)propanoic acid,
2(S)-3-(4-{2-[6-(cyclohexylcarbonyl)-1H-pyrrolo[2,3-b]pyridin-1-yl]ethoxy}phenyl)-2-(2,2,2-trifluoroethoxy)propanoic acid,
methyl 3-(4-{2-[6-(cyclopentylcarbonyl)-1H-pyrrolo[2,3-b]pyridin-1-yl]ethoxy}-phenyl)-2-ethoxypropanoate,
3-(4-{2-[6-(cyclopentylcarbonyl)-1H-pyrrolo[2,3-b]pyridin-1-yl]ethoxy}phenyl)-2-ethoxypropanoic acid,
3-[4-(2-{6-[cyclopropyl(hydroxyimino)methyl]-1H-pyrrolo[2,3-b]pyridin-1-yl}ethoxy)-phenyl]-2-(2,2,2-trifluoroethoxy)propanoic acid,
2-ethoxy-3-[4-(2-{6-[(hydroxyimino) (phenyl)methyl]-1H-pyrrolo[2,3-b]pyridin-1-yl}-ethoxy)phenyl]propanoic acid,
3-[4-(2-{6-[cyclopropyl(hydroxyimino)methyl]-1H-pyrrolo[2,3-b]pyridin-1-yl}ethoxy)-phenyl]-2-ethoxypropanoic acid.

The compounds of the present invention have very valuable pharmacological properties.

The compounds demonstrate especially an excellent activity in lowering blood glucose levels. As a result of such properties they can be used therapeutically in the treatment and/or prophylaxis of hyperglycaemia, dyslipidaemia and, more especially, in the treatment of non-insulin-dependent type II diabetes, glucose intolerance, disorders associated with syndrome X (including hypertension, obesity, insulin resistance, atherosclerosis, hyperlipidaemia), coronary artery disease and other cardiovascular diseases (including arterial hypertension, cardiac insufficiency, venous insufficiency), renal disorders (including glomerulonephritis, glomerulosclerosis, nephrotic syndrome, hypertensive nephrosclerosis), retinopathy, disorders associated with the activation of endothelial cells, psoriasis, polycystic ovary syndrome, dementia, diabetic complications and osteoporosis.

They can be used as aldose reductase inhibitors, for improving cognitive functions in dementia and for the complications of diabetes, intestinal inflammatory disorders, myotonic dystrophy, pancreatitis, arteriosclerosis, xanthoma.

The activity of these compounds is also recommended for the treatment and/or prophylaxis of other diseases, including type I diabetes, hypertriglyceridaemia, syndrome X, insulin resistance, dyslipidaemia in diabetics, hyperlipidaemia, hypercholesterolaemia, arterial hypertension, cardiac insufficiency, and cardiovascular disease, especially atherosclerosis.

The compounds are furthermore indicated for use in the regulation of appetite, especially in the regulation of food intake in subjects suffering from disorders such as obesity, anorexia, bulimia and anorexia nervosa.

The compounds can accordingly be used in the prevention or treatment of hypercholesterolaemia, obesity with advantageous effects on hyperlipidaemia, hyperglycaemia, osteoporosis, glucose intolerance, insulin resistance or disorders in which insulin resistance is a secondary physiopathological mechanism.

The use of those compounds enables reduction of total cholesterol, body weight, leptin resistance, plasma glucose, triglycerides, LDLs, VLDLs and also plasma free fatty acids. The compounds can be used in association with HMG CoA reductase inhibitors, fibrates, nicotinic acid, cholestyramine, colestipol, probucol, GLP1, metformin, the biguanides or glucose reabsorption inhibitors and can be administered together or at different times to act in synergy in the patient treated.

They furthermore exhibit activity in cancer pathologies and especially hormone-dependent cancers, such as breast cancer and colon cancer, and also have an inhibiting effect on the angiogenesis processes implicated in those pathologies.

Amongst the pharmaceutical compositions according to the invention there may mentioned more especially those which are suitable for oral, parenteral, nasal, per- or trans-cutaneous, rectal, perlingual, ocular or respiratory administration and especially tablets or dragées, sublingual tablets, sachets, paquets, gelatin capsules, glossettes, lozenges, suppositories, creams, ointments, dermal gels and drinkable or injectable ampoules.

The dosage varies in accordance with the sex, age and weight of the patient, the administration route, the nature of the therapeutic indication or of any associated treatments and ranges from 0.1 mg to 1 g per 24 hours taken in 1 or more administrations.

The present invention relates also to a new association between a heterocyclic compound of formula (I) or (V) as defined hereinbefore and an antioxidant agent for obtaining pharmaceutical compositions for use in the treatment and/or prevention of obesity and overweight characterised by a body mass index greater than 25.

The antioxidant agents according to the invention are, more specifically, anti-free radical agents or free-radical trapping agents, antilipoperoxidant agents, chelating agents or agents capable of regenerating endogenous antioxidants such as glutathione, vitamin C or vitamin E, and also addition salts thereof with a pharmaceutically acceptable acid or base.

The antioxidant agent of the association according to the invention is more preferably represented by quinone compounds such as ubiquinone or coenzyme Q₁₀, which acts as a free-radical trapping agent but which is also capable of regenerating vitamin E.

The preferred association according to the invention is (2S)-3-[4-(2-{6-[(methoxyimino)-(phenyl)methyl]-1H-pyrrolo[2,3-b]pyridin-1-yl}ethoxy)phenyl]-2-(2,2,2-trifluoroethoxy)-propanoic acid and coenzyme Q₁₀.

Furthermore, the association according to the invention has entirely surprising pharmacological properties: the Applicant has demonstrated that a synergy exists between the two compounds of the association allowing a very significant reduction in body fat to be obtained, making it useful in the treatment and/or prevention of obesity and of overweight characterised by a body mass index greater than 25.

In the United States, obesity affects 20% of men and 25% of women. Patients having a body mass index (BMI=weight (kg)/height² (m²)) greater than or equal to 30 are considered to be obese (Int. J. Obes., 1998, 22, 39-47; Obesity Lancet, 1997, 350, 423-426). Obesity (BMI≧30) and overweight (25<BMI <30) can have various origins: they may come about following deregulation of food intake, following hormonal disturbance, or following administration of a treatment: treating type II diabetes with sulphonylureas causes patients to gain weight. Similarly, in type I (insulin-dependent) diabetes, insulin therapy is also a cause of weight gain in patients (In Progress in Obesity Research, 8^{th} International Congress on Obesity, 1999, 739-746; Annals of Internal Medicine, 1998, 128, 165-175).

Obesity and overweight are well-established risk factors for cardiovascular diseases: they are associated with a significant increase in the risk of cerebro-vascular accidents and non-insulin-dependent diabetes, because they predispose to insulin resistance, to dyslipidaemia and to the appearance of macrovascular disorders (nephropathy, retinopathy, angiopathy). Further pathologies are the consequence of obesity or overweight: there may be mentioned, in particular, vesicular calculi, respiratory dysfunction, several forms of cancer and, in the case of very severe obesity, premature death (N. Engl. J. Med., 1995, 333, 677-385; JAMA, 1993, 270, 2207-2212).

The association according to the invention allows a weight loss to be obtained which, even if moderate, significantly reduces all the risk factors associated with obesity (Int. J. Obes., 1997, 21, 55-9; Int. J. Obes., 1992, 21, S5-9).

The association according to the invention will therefore be found to be useful in the treatment and/or prevention of obesity and of overweight characterised by a body mass index greater than 25.

The invention accordingly relates to the use of the association between a compound of formula (I) or (V) and an antioxidant agent in obtaining pharmaceutical compositions intended for the treatment and/or prevention of obesity and of overweight characterised by a body mass index greater than 25 and less than 30.

In particular, the association according to the invention is useful in the treatment and/or prevention of obesity and of overweight characterised by a body mass index greater than 25 and less than 30 induced by therapeutic treatment, such as treatment for type I or type II diabetes.

The invention accordingly relates to the use of the association between a compound of formula (I) or (V) and an antioxidant agent in obtaining pharmaceutical compositions intended for the treatment and/or prevention of obesity and of overweight characterised by a body mass index greater than 25 and less than 30 induced by therapeutic treatment, such as treatment for type I or type II diabetes.

The invention relates also to pharmaceutical compositions comprising the association between a compound of formula (I) or (V) and an antioxidant agent, as defined hereinbefore, in combination with one or more pharmaceutically acceptable excipients.

Among the pharmaceutical compositions according to the invention there may be mentioned, more especially, those that are suitable for oral, parenteral or nasal administration, tablets or dragees, sublingual tablets, capsules, lozenges, suppositories, creams, ointments, dermal gels, etc.

In particular, the invention relates to pharmaceutical compositions comprising a compound of formula (I) or (V) as defined hereinbefore and an antioxidant agent, such as coenzyme Q₁₀ or vitamin E, in combination with one or more pharmaceutically acceptable excipients.

The dosage varies according to the sex, age and weight of the patient, the administration route, the nature of the therapeutic indication or of any associated treatments and ranges from 0.1 mg to 1 g of each component of the association per 24 hours in one or more administrations.

The Examples and Preparations which follow illustrate the invention but do not limit it in any way.

### Preparation 1: Methyl 3-(4-{2-[6-(cyclopropylcarbonyl)-1H-pyrrolo[2,3-b]pyridin-1-yl]ethoxy}phenyl)-2-ethoxypropanoate

### Step A: Cyclopropyl(1H-pyrrolo[2,3-b]pyridin-6-yl)methanone

In a round-bottomed flask under an argon atmosphere, 3 g (21 mmol) of 1H-pyrrolo[2,3-b]pyridine-6-carbonitrile are dissolved in 35 ml of anhydrous tetrahydrofuran. 5.35 ml (42 mmol) of trimethylsilyl chloride and 126 ml (126 mmol) of phenylmagnesium bromide (1M in tetrahydrofuran) are then added. The reaction mixture is stirred for 1 night at ambient temperature. It is then hydrolysed with 100 ml of 2M ammonium chloride solution. The pH is then acidified to 1 using 10% hydrochloric acid and the mixture is stirred for 2 hours at ambient temperature. The pH of that solution is then brought to 9 using concentrated ammonium hydroxide solution. The aqueous phase is extracted twice with 100 ml of dichloromethane. The organic phases are dried over magnesium sulphate, filtered and evaporated under reduced pressure.

The crude product is purified by chromatography on silica gel (petroleum ether/ethyl acetate: 9/1 then 8/2) to yield the title product in the form of a beige solid.

Melting point: 159° C.

MS: m/z=187 [M+H]⁺.

### Step B: Methyl 3-(4-{2-[6-(cyclopropylcarbonyl)-1H-pyrrolo[2,3-b]pyridin-1-yl]ethoxy}phenyl)-2-ethoxypropanoate

In a round-bottomed flask under argon, 0.500 g (2.68 mmol) of the compound obtained in Step A are dissolved in 30 ml of anhydrous N,N-dimethylformamide. 0.161 g (4.02 mmol) of sodium hydride are then added. That mixture is stirred for 1 hour at ambient temperature and cooled to 0° C. The solution is then added to 1 g (3.48 mmol) of methyl 3-[4-(2-bromoethoxy)phenyl]-2-ethoxypropanoate dissolved in N,N-dimethylformamide. The reaction mixture is stirred for 5 hours at ambient temperature. It is then hydrolysed with 50 ml of saturated ammonium chloride solution. The aqueous phase is extracted 3 times with 50 ml of dichloromethane. The organic phases are dried over magnesium sulphate, filtered and evaporated under reduced pressure. The crude product obtained is purified by chromatography on silica gel (petroleum ether/ethyl acetate: 9/1 then 8/2) to yield the title product in the form of a yellow solid.

Melting point: 159° C.

MS: m/z=437 [M+H]⁺.

### Preparation 2: Methyl 3-(4-{2-[6-(cyclopropylcarbonyl)-1H-pyrrolo[2,3-b]pyridin-1-yl]ethoxy}phenyl)-2-(2,2,2-trifluoroethoxy)propanoate

In a two-necked flask under an inert atmosphere, the compound obtained in Step A of Preparation 1 (0.600 g) is dissolved in 30 ml of anhydrous N,N-dimethylformamide, and then 0.193 g (5.31 mmol) of sodium hydride (60% in oil) is added. The mixture is stirred for 1 hour and 30 minutes at ambient temperature and cooled to 0° C. The anion obtained is then added to 0.485 g of methyl 3-[4-(2-bromoethoxy)phenyl]-2-(2,2,2-trifluoroethoxy)propanoate (1.26 mmol) dissolved in 15 ml of anhydrous N,N-dimethylformamide. The reaction mixture is stirred for 7 hours at ambient temperature. The solvent is then evaporated off and the residue is taken up in 40 ml of water. It is extracted 3 times with 40 ml of ethyl acetate. The combined organic phases are dried over magnesium sulphate, filtered and evaporated under reduced pressure. The crude product is purified by chromatography on silica gel (petroleum ether/ethyl acetate: 10/0, 9/1 then 8/2) to yield the title product in the form of a red solid.

Melting point: 123° C.

MS: m/z=513 [M+Na]⁺.

### Preparation 3: Methyl 3-(4-{2-[6-(cyclohexylcarbonyl)-1H-pyrrolo[2,3-b]pyridin-1-yl]ethoxy}phenyl)-2-ethoxypropanoate

### Step A: 6-(1-Cyclohexylvinyl)-1H-pyrrolo[2,3-b]pyridine

Under an argon atmosphere, 1.5 g (10.48 mmol) of 1H-pyrrolo[2,3-b]pyridine-6-carbonitrile are dissolved in 20 ml of anhydrous tetrahydrofuran. 2.66 ml (20.96 mmol) of trimethysilyl chloride are then added. That mixture is then added to 31 ml (62.87 mmol) of cyclohexylmagnesium chloride dissolved in 20 ml of anhydrous tetrahydrofuran. The reaction mixture is stirred for 20 hours at ambient temperature. It is then hydrolysed with 50 ml of 2M ammonium chloride. After acidification to pH 1 using 6M hydrochloric acid, the mixture is stirred for 2 hours at ambient temperature. It is then rendered basic to pH 8 using aqueous ammonium hydroxide solution and extracted 3 times with 20 ml of dichloromethane. The organic phases are dried over magnesium sulphate, filtered and evaporated.

The crude product is purified by chromatography on silica gel (petroleum ether/ethyl acetate: 9/1, 8/2 then 7/3) to yield the title product in the form of a yellowish solid.

Melting point: 160° C.

MS: m/z=229 [M+H]⁺.

### Step B: Methyl 3-(4-{2-[6-(cyclohexylcarbonyl)-1H-pyrrolo[2,3-b]pyridin-1-yl]-ethoxy}phenyl)-2-ethoxypropanoate

In a two-necked flask under argon; 0.376 g (1.65 mmol) of the compound obtained in Step A are dissolved in 20 ml of anhydrous N,N-dimethylformamide and 0.091 g (3.79 mmol) of sodium hydride (60% in oil) are added. The mixture is stirred for 1 hour at ambient temperature. It is then added to a solution containing 0.616 g (2.15 mmol) of methyl 3-[4-(2-bromoethoxy)phenyl]-2-ethoxypropanoate and 10 ml of anhydrous N,N-dimethylformamide. The reaction mixture is stirred for 4 hours at ambient temperature. It is then hydrolysed with 30 ml of saturated ammonium chloride solution and extracted 3 times with 30 ml of ethyl acetate. The organic phases are washed with 50 ml of saturated sodium chloride solution, dried over magnesium sulphate, filtered and evaporated under reduced pressure to yield the title product in the form of a yellow oil.

MS: niz=479 [M+H]⁺.

### Preparation 4: Methyl (2S)-3-(4-{2-[6-(cyclohexylcarbonyl)-1H-pyrrolo[2,3-b]-pyridin-1-yl]ethoxy}phenyl)-2-ethoxypropanoate

The procedure is as in Preparation 3, with the replacement of methyl 3-[4-(2-bromo-ethoxy)phenyl]-2-ethoxypropanoate with methyl (2S)-3-[4-(2-bromoethoxy)phenyl]-2-ethoxypropanoate in Step B.

[α]_{D}=−7 (methanol, c=1).

### Preparation 5: Methyl 3-(4-{2[6-(cyclohexylcarbonyl)-1H-pyrrolo[2,3-b]pyridin-1-yl]ethoxy}phenyl)-2-(2,2,2-trifluoroethoxy)propanoate

The procedure is as in Preparation 2, starting from Step A of Preparation 3. Yellow oil.

MS: m/z=533 [M+H]⁺.

### Preparation 6: Methyl (2S)-3-(4-{2-[6-(cyclohexylcarbonyl)-1H-pyrrolo[2,3-b]pyridin-1-yl]ethoxy}phenyl)-2-(2,2,2-trifluoroethoxy)propanoate

The procedure is as in Preparation 3, with the replacement of methyl 3-[4-(2-bromo-ethoxy)phenyl]-2-ethoxypropanoate with methyl (2S)-3-[4-(2-bromoethoxy)phenyl]-2-(2,2,2-trifluoroethoxy)propanoate in Step B.

[α]_{D}=−15 (methanol, c=1).

### Preparation 7: 3-(4-{2-[6-(Cyclopropylcarbonyl)-1H-pyrrolo[2,3-b]pyridin-1-yl]ethoxy}phenyl)-2-ethoxypropanoic acid

The compound obtained in Preparation 1 (0.340 g) is dissolved in 20 ml of tetrahydrofuran. 0.102 g (2.34 mmol) of lithium hydroxide dissolved in 20 ml of water is then added. The mixture is stirred for 2 hours at ambient temperature. The solvent is then evaporated off and the residue is acidified to pH 3 using aqueous acetic acid. The aqueous phase is extracted 3 times with dichloromethane. The organic phases are dried over magnesium sulphate, filtered and evaporated under reduced pressure. The crude product is purified by chromatography on silica gel (dichloromethane/methanol:97/3) to yield the title product in the form of a yellow gum.

MS: m/z=423 [M+H]⁺.

### Preparation 8: 3-(4-{2-[6-(Cyclopropylcarbonyl)-1H-pyrrolo[2,3-b]pyridin-1-yl]ethoxy}phenyl)-2-(2,2,2-trifluoroethoxy)propanoic acid

The compound obtained in Preparation 2 (0.200 g) is dissolved in tetrahydrofuran and cooled to 0° C. 0.052 g (1.23 mmol) of lithium hydroxide monohydrate dissolved in 10 ml of water is then added. The reaction mixture is stirred for 1 hour and 30 minutes from 0° C. to ambient temperature. The solvent is evaporated off and the white residue obtained is dissolved in 20 ml of water. After acidification to pH 3 using aqueous acetic acid, extraction is carried out 3 times with 10 ml of dichloromethane. The organic phases are dried over magnesium sulphate, filtered and evaporated under reduced pressure. The crude product is purified by chromatography on silica gel (dichloromethane/methanol:95/5) to yield the title product in the form of a white solid.

Melting point: 113° C.

MS: m/z=475 [M−H]⁺.

### Preparation 9: 3-(4-{2-[6-(Cyclohexylcarbonyl)-1H-pyrrolo[2,3-b]pyridin-1-yl]ethoxy}phenyl)-2-ethoxypropanoic acid

In a round-bottomed flask under an argon atmosphere, 0.200 g (0.42 mmol) of the compound obtained in Preparation 3 is dissolved in 30 ml of a 50/50 water/tetrahydrofuran mixture. 53 mg (1.26 mmol) of lithium hydroxide are then added. The reaction mixture is stirred for 1 hour at ambient temperature. The tetrahydrofuran is then evaporated off under reduced pressure. The residue is acidified to pH 3 using aqueous acetic acid and extracted 3 times with 20 ml of ethyl acetate. The organic phases are dried over magnesium sulphate, filtered and evaporated under reduced pressure. The residue is purified by chromatography on silica gel (pure dichloromethane then dichloromethane/methanol:95/5 then 90/10) to yield the title product in the form of a yellow oil.

MS m/z=465 [M+H]⁺.

### Preparation 10: (2S)-3-(4-{2-[6-(Cyclohexylcarbonyl)-1H-pyrrolo[2,3-b]pyridin-1-yl]ethoxy}phenyl)-2-ethoxypropanoic acid

In a round-bottomed flask, 0.180 g (0.38 mmol) of the compound obtained in Preparation 4 is dissolved in 4 ml of tetrahydrofuran and cooled to 0° C. 0.048 g (1.14 mmol) of lithium hydroxide dissolved in 7 ml of water is then added. The reaction mixture is stirred for 9 hours from 0° C. to ambient temperature. The solvent is evaporated off. The residue is washed with 10 ml of diethyl ether. The aqueous phase is acidified to pH 3 using acetic acid. It is extracted 3 times with 10 ml of ether. The organic phases are washed with 20 ml of saturated sodium chloride solution, dried over magnesium sulphate, filtered and evaporated under reduced pressure to yield the title product in the form of an oil.

[α]_{D}=−11 (methanol, c=1).

CE: purity≧99%; ee≧98%.

### Preparation 11: 3-(4-{2-[6-(Cyclohexylcarbonyl)-1H-pyrrolo[2,3-b]pyridin-1-yl]ethoxy}phenyl)-2-(2,2,2-trifluoroethoxy)propanoic acid

The procedure is as in Preparation 9, starting from the compound obtained in Preparation 5. Yellow oil.

MS: m/z=519 [M+H]⁺.

### Preparation 12: 2(S)-3-(4-{2-[6-(Cyclohexylcarbonyl)-1H-pyrrolo[2,3-b]pyridin-1-yl]ethoxy}phenyl)-2-(2,2,2-trifluoroethoxy)propanoic acid

The procedure is as in Preparation 9, starting from the compound obtained in Preparation 6. Oil.

[α]_{D}=−11 (methanol, c=1).

CE: purity≧99%; ee≧98%.

### Preparation 13: Methyl 3-(4-{2-[6-(cyclopentylcarbonyl)-1H-pyrrolo[2,3-b]pyridin-1-yl]ethoxy}phenyl)-2-ethoxypropanoate

The procedure is as in Preparation 3, with the replacement of cyclohexylmagnesium chloride with cyclopentylmagnesium chloride in Step A.

### Preparation 14: 3-(4-{2-[6-(Cyclopentylcarbonyl)-1H-pyrrolo[2,3-b]pyridin-1-yl]ethoxy}phenyl)-2-ethoxypropanoic acid

The procedure is as in Preparation 9, starting from the compound obtained in Preparation 13.

### EXAMPLE 1

### Methyl 2-ethoxy-3-[4-(2-{6-[(E)-(methoxyimino)(phenyl)methyl]-1H-pyrrolo[2,3-b]pyridin-1-yl}ethoxy)phenyl]propanoate

### Step A: Phenyl(1H-pyrrolo[2,3-b]pyridin-6-yl)methanone

In a round-bottomed flask under argon, 25.2 ml of 1M phenylmagnesium bromide in tetrahydrofuran are dissolved in 7 ml of anhydrous tetrahydrofuran. In another round-bottomed flask under argon, 0.6 g of 1H-pyrrolo[2,3-b]pyridine-6-carbonitrile and 1.07 ml of trimethylsilyl chloride are dissolved in 7 ml of tetrahydrofuran. That solution is then pipetted onto the magnesium solution at ambient temperature. After stirring for one night, the mixture is hydrolysed with 20 ml of 2N ammonium chloride solution. The pH is then adjusted to 1 using 10% hydrochloric acid and the solution obtained is stirred for 2 hours at ambient temperature. The pH of the solution is adjusted to 9 using concentrated ammonium hydroxide solution and the aqueous phase is extracted twice with 20 ml of dichloromethane. The organic phases are dried over magnesium sulphate, filtered and evaporated under reduced pressure. After purification by chromatography on silica gel (pure dichloromethane and then dichloromethane/methanol:99/1), the title compound is obtained in the form of a yellow solid.

Melting point: 174° C.

### Step B: (E)-Phenyl(1H-pyrrolo[2,3-b]pyridin-6-yl)methanone O-methyloxime

In a round-bottomed flask under an argon atmosphere, 4.5 g of the compound obtained in Step A, 2.26 g of methoxyammonium chloride and 2.2 ml of pyridine are dissolved in 200 ml of absolute ethanol. The reaction mixture is stirred for 24 hours at ambient temperature. The solvent is then evaporated off and the residue is taken up in 150 ml of water. The aqueous phase is extracted 3 times with 100 ml of dichloromethane. The organic phases are dried over magnesium sulphate, filtered and evaporated under reduced pressure. The crude product is purified by chromatography on silica gel (petroleum ether/ethyl acetate: 5/1). The two isomers, Z and E, are in that manner separated and isolated.

E isomer: white solid.

Melting point: 158° C.

### Step C: Methyl 2-ethoxy-3-[4-(2-{6-[(E)-(methoxyimino) (phenyl)methyl]-1H-pyrrolo[2,3-b]pyridin-1-yl}yethoxy)phenyl]propanoate

In a round-bottomed flask under an inert atmosphere, 0.400 g of the compound obtained in Step B are dissolved in 16 ml of anhydrous N,N-dimethylformamide. 0.096 g of sodium hydride (60% in oil) is then added and the mixture is stirred for 1 hour at ambient temperature. That solution is then added to 0.690 g of methyl 3-[4-(2-bromoethoxy)phenyl]2-ethoxypropanoate dissolved in 8 ml of N,N-dimethylformamide. The reaction mixture is stirred for 4 hours at ambient temperature. It is then hydrolysed with 30 ml of saturated ammonium chloride solution. The aqueous phase is extracted twice with 30 ml of dichloromethane. The organic phases are dried over magnesium sulphate, filtered and evaporated under reduced pressure. The crude product obtained is purified by chromatography on silica gel (petroleum ether/ethyl acetate:8/2) and yields the title product in the form of a yellow oil.

MS: m/z=502 [M+H]⁺.

### EXAMPLE 2

### Methyl (2S)-2-ethoxy-3-[4-(2-{6-[(E)-(methoxyimino)(phenyl)methyl]-1H-pyrrolo[2,3-b]pyridin-1-yl}ethoxy)phenyl]propanoate

The procedure is as in Example 1, with the replacement of methyl 3-[4-(2-bromoethoxy)phenyl]-2-ethoxypropanoate with methyl (2S)-3-[4-(2-bromoethoxy)phenyl]-2-ethoxypropanoate in Step C.

[α]_{D}=−5 (methanol, c=1).

### EXAMPLE 3

### Methyl 3-[4-(2-{6-[(E)-(methoxyimino)(phenyl)methyl]-1H-pyrrolo[2,3-b]pyridin-1-yl}ethoxy)phenyl]-2-(2,2,2-trifluoro-ethoxy)propanoate

The procedure is as in Example 1, with the replacement of methyl 3-[4-(2-bromo-ethoxy)phenyl]-2-ethoxypropanoate with methyl 3-[4-(2-bromoethoxy)phenyl]-2-(2,2,2-trifluoroethoxy)propanoate in Step C.

### EXAMPLE 4

### Methyl (2S)-3-[4-(2-{6-[(E)-(methoxyimino)(phenyl)methyl]-1H-pyrrolo[2,3-b]pyridin-1-yl}ethoxy)phenyl]-2-(2,2,2-trifluoro-ethoxy)propanoate

The procedure is as in Example 1, with the replacement of methyl 3-[4-(2-bromo-ethoxy)phenyl]-2-ethoxypropanoate with methyl (2S)-3-[4-(2-bromoethoxy)phenyl]-2-(2,2,2-trifluoroethoxy)propanoate in Step C.

### EXAMPLE 5

### 2-Ethoxy-3-[4-(2-{6-[(E)-(methoxyimino)(phenyl)methyl]-1H-pyrrolo[2,3-b]pyridin-1-yl}ethoxy)phenyl]propanoic acid

The compound obtained in Example 1 (0.360 g) is dissolved in 20 ml of a 50/50 water/tetrahydrofuran mixture. 0.094 g of lithium hydroxide is then added. The reaction mixture is stirred for 2 hours at ambient temperature. The tetrahydrofuran is then evaporated off and the residue obtained is acidified to pH 3 using aqueous acetic acid. That aqueous phase is extracted 3 times with 50 ml of dichloromethane. The combined organic phases are dried over magnesium sulphate, filtered and evaporated under reduced pressure. The crude product is purified by chromatography on silica gel (dichloromethane/methanol 97/3) and yields the title product in the form of a slightly yellow gum.

MS: m/z=488 [M+H]⁺.

### EXAMPLE 6

### (2S)-2-Ethoxy-3-[4-(2-{6-[(E)-(methoxyimino)(phenyl)methyl]-1H-pyrrolo[2,3-b]pyridin-1-yl}ethoxy)phenyl]propanoic acid

The compound obtained in Example 2 (0.140 g) is dissolved in 3 ml of tetrahydrofuran and cooled to 0° C. 0.035 g (0.84 mmol) of lithium hydroxide monohydrate dissolved in 5 ml of water is then added. The reaction mixture is stirred for 3 hours from 0° C. to ambient temperature. The solvent is evaporated off. The residue obtained is washed with 10 ml of diethyl ether. The aqueous phase is acidified to pH 3 using aqueous acetic acid. It is then extracted 3 times with 10 ml of ether. The organic phases are dried over magnesium sulphate, filtered and evaporated under reduced pressure. The residue is purified by chromatography on silica gel (dichloromethane/methanol:95/5).

[α]_{D}=−10 (methanol, c=1).

CE: purity≧99%; ee≧98%.

### EXAMPLE 7

### 3-[4-(2-{6-[(E)-(Methoxyimino)(phenyl)methyl]-1H-pyrrolo[2,3-b]pyridin-1-yl}ethoxy)phenyl]-2-(2,2,2-trifluoroethoxy)propanoic acid

The title acid is obtained in the form of a yellow mousse in accordance with the procedure used for Example 6, starting from the compound obtained in Example 5 (0.900 g).

MS: m/z=541 [M+H]⁺.

### EXAMPLE 8

### (2S)-3-[4-(2-{6-[(E)-(Methoxyimino)(phenyl)methyl]-1H-pyrrolo[2,3-b]pyridin-1-yl}ethoxy)phenyl]-2-(2,2,2-trifluoroethoxy)propanoic-acid

In a round-bottomed flask, 0.070 g (0.13 mmol) of the compound obtained in Example 4 is dissolved in 1.5 ml of tetrahydrofuran and cooled to 0° C. 0.016 g (0.39 mmol) of lithium hydroxide dissolved in 2.5 ml of water is then added. The reaction mixture is stirred for 2 hours at ambient temperature. The solvent is evaporated off and then the residue is acidified to pH 3 using acetic acid. The aqueous phase is extracted 3 times with 10 ml of ether. The organic phases are dried over magnesium sulphate, filtered and evaporated under reduced pressure.

The crude product is purified by chromatography on silica gel (dichloromethane/methanol 95/5).

[α]_{D}=−15 (methanol, c=1).

CE: purity≧99%; ee≧98%.

### EXAMPLE 9

### Methyl 2-ethoxy-3-[4-(2-{6-[(Z)-(methoxyimino)(phenyl)methyl]-1H-pyrrolo[2,3-b]pyridin-1-yl}ethoxy)phenyl]propanoate

Step A: (Z)-Phenyl(1H-pyrrolo[2,3-b]pyridin-6-yl)methanone O-methyloxime In a round-bottomed flask under an argon atmosphere, 4.5 g of the compound obtained in Step A of Example 1, 2.26 g of methoxyammonium chloride and 2.2 ml of pyridine are dissolved in 200 ml of absolute ethanol. The reaction mixture is stirred for 24 hours at ambient temperature. The solvent is then evaporated off and the residue is taken up in 150 ml of water. The aqueous phase is extracted 3 times with 100 ml of dichloromethane. The organic phases are dried over magnesium sulphate, filtered and evaporated under reduced pressure. The crude product is purified by chromatography on silica gel (petroleum ether/ethyl acetate:5/1). The two isomers, Z and E, are in that manner separated and isolated.

Z isomer: white solid.

Melting point: 189° C.

### Step B: Methyl 2-ethoxy-3-[4-(2-(6-[(Z)-(methoxyimino)(phenyl)methyl]-1H-pyrrolo[2,3-b]pyridin-1-yl]ethoxy)phenylypropanoate

The procedure is as in Step C of Example 1.

Yellow oil

MS: m/z=502 [M+H]⁺.

### EXAMPLE 10

### 2-Ethoxy-3-[4-(2-{6-[(Z)-(methoxyimino)(phenyl)methyl]-1H-pyrrolo[2,3-b]pyridin-1-yl}ethoxy)phenyl]propanoic acid

The title acid is obtained starting from the compound obtained in Example 9 in accordance with the procedure of Example 5.

Yellow gum

MS: m/z=488 [M+H]⁺.

### EXAMPLE 11

### Methyl 2-ethoxy-3-[4-(2-{5-[(methoxyimino)(phenyl)methyl]-1H-pyrrolo[2,3-b]pyridin-1-yl}ethoxy)phenyl]propanoate

### Step A Phenyl(1H-pyrrolo[2,3-b]pyridin-5-yl)methanone

In a round-bottomed flask, 31 mg (0.31 mmol) of chromium trioxide are dissolved in 140 ml of dichloromethane and then 3.43 ml (24.99 mmol) of tert-butanol hydroperoxide (70% in water) are slowly added. The solution turns deep red. Phenyl(1H-pyrrolo[2,4-b]pyridin-5-yl)methanol (1.4 g) is slowly added. The reaction mixture, which turns yellow, is stirred for 21 hours at ambient temperature in the open air. The peroxides are neutralised with 30 ml of sodium bisulphite solution and the mixture is extracted 3 times with 50 ml of dichloromethane. The organic phases are dried over magnesium sulphate, filtered and then evaporated under reduced pressure.

The residue is purified by chromatography on silica gel (petroleum ether/ethyl acetate: 6/4 then 5/5) to yield the title product in the form of a pink solid.

Melting point: 174° C.

MS: m/z=223 [M+H]⁺.

### Step B: Phenyl(1H-pyrrolo[2,3-b]pyridin-5-yl)methanone O-methyloxime

In a round-bottomed flask under an inert atmosphere, 1 g (4.50 mmol) of the compound obtained in Step A is dissolved in 40 ml of absolute ethanol. 0.55 ml (6.75 mmol) of pyridine and 0.564 g (6.75 mmol) O-methylhydroxylamine hydrochloride are then added. The reaction mixture is stirred for 15 hours at reflux and then allowed to return to ambient temperature. The ethanol is evaporated off under reduced pressure, and the residue is taken up in a water/dichloromethane mixture (50 ml). It is extracted 3 times with 50 ml of dichloromethane and then the organic phases are dried over magnesium sulphate, filtered and evaporated.

Chromatography on silica gel (petroleum ether/ethyl acetate:6/4) allows a mixture of the 2 expected isomers to be obtained in a 50/50 ratio in the form of a white solid

Melting point: 145° C.

MS: m/z=252 [M+H]⁺.

### Step C: Methyl 2-ethoxy-3-[4-(2-{5-[(methoxyimino)(phenyl)methyl]-1H-pyrrolo[2,3-b]pyridin-1-yl}ethoxy)phenyl]propanoate

In a round-bottomed flask under an argon atmosphere, 0.800 g (3.18 mmol) of the compound obtained in Step B is dissolved in 15 ml of N,N-dimethylformamide, and then 0.191 g (4.77 mmol) of sodium hydride (60% in oil) is added. The mixture is stirred for an hour and a half at ambient temperature. The anion formed is then cooled to 0° C. It is then transferred to a mixture composed of 52 mg (10 mol %) of potassium iodide, 1.47 g (3.82 mmol) of methyl 3-[4-(2-bromoethoxy)phenyl]-2-ethoxypropanoate and 25 ml of N,N-dimethylformamide. The reaction mixture is stirred for 2 hours from 0° C. to ambient temperature. The solvent is then evaporated off. The residue is taken up in 20 ml of water and extracted with ethyl acetate (3 times, 20 ml each time). The organic phases are washed with 30 ml of brine, dried over magnesium sulphate, filtered and evaporated. Chromatography on silica gel (petroleum ether/ethyl acetate: 7/3 then 6/4) allows the title product to be obtained in a mixture of the 2 isomers in a Z/E proportion of 50/50 in the form of a yellow oil.

MS: m/z=502 [M+H]⁺.

### EXAMPLE 12

### Methyl 3-[4-(2-{5-[(methoxyimino)(phenyl)methyl]-1H-pyrrolo[2,3-b]pyridin-1-yl}ethoxy)phenyl]-2-(2,2,2-trifluoroethoxy)propanoate

In a round-bottomed flask under an argon atmosphere, 0.800 g (3.18 mmol) of the compound obtained in Step B of Example 11 is dissolved in 15 ml of anhydrous N,N-dimethylformamide, and then 0.191 g (4.77 mmol) of sodium hydride (60% in oil) is added. The mixture is stirred for an hour and a half at ambient temperature. The anion formed is then cooled to 0° C. It is subsequently transferred to a mixture composed of 52 mg (10 mol %) of potassium iodide, 1.47 g (3.82 mmol) of methyl 3-[4-(2-bromoethoxy)-phenyl]-2-(2,2,2-trifluoroethoxy)propanoate and 25 ml of anhydrous N,N-dimethyl-formamide. The reaction mixture is stirred for 2 hours from 0° C. to ambient temperature. The solvent is then evaporated off. The residue is taken up in 20 ml of water and extracted with ethyl acetate (3 times, 20 ml each time). The organic phases are washed with 30 ml of saturated sodium chloride solution, dried over magnesium sulphate, filtered and evaporated. Chromatography on silica gel (petroleum ether/ethyl acetate: 7/3 then 6/4) allows the title product to be obtained in a mixture of the 2 isomers in a Z/E proportion of 50/50 in the form of a yellow oil.

MS: m/z=556 [M+H]⁺.

### EXAMPLE 13

### 2-Ethoxy-3-[4-(2-{5-[(methoxyimino)(phenyl)methyl]-1H-pyrrolo[2,3-b]pyridin-1-yl}ethoxy)phenyl]propanoic acid

The compound obtained in Example 11 (0.500 g) is dissolved in 10 ml of tetrahydrofuran and cooled to 0° C. 0.113 g (2.70 mmol) of lithium hydroxide monohydrate dissolved in 17 ml of water is then added. The reaction mixture is stirred for 2 hours at 0° C. and then the solvent is evaporated off. The residue is taken up in 10 ml of water and acidified to pH 3 using aqueous acetic acid. It is extracted 3 times with 10 ml of dichloromethane. The organic phases are dried over magnesium sulphate, filtered and evaporated. The residue is purified by chromatography on silica gel (dichloromethane/methanol:9/1) and the title product is obtained in the form of a white gum.

MS: m/z=488 [M+H]⁺.

### EXAMPLE 14

### 3-[4-(2-{5-[(Methoxyimino)(phenyl)methyl]-1H-pyrrolo[2,3-b]pyridin-1-yl}ethoxy)phenyl]-2-(2,2,2-trifluoroethoxy)propanoic acid

The compound obtained in Example 12 (0.500 g) is dissolved in 10 ml of tetrahydrofuran and cooled to 0° C. 0.113 g (2.70 mmol) of lithium hydroxide monohydrate dissolved in 17 ml of water is then added. The reaction mixture is stirred for 2 hours at 0° C. and then the solvent is evaporated off. The residue is taken up in 10 ml of water and acidified to pH 3 using aqueous acetic acid. It is extracted 3 times with 10 ml of dichloromethane. The organic phases are dried over magnesium sulphate, filtered and evaporated. The residue is purified by chromatography on silica gel (dichloromethane/methanol:9/1) to yield the title product in the form of a white solid.

Melting point: 182° C. (degradation).

MS: m/z=542 [M+H]⁺.

### EXAMPLE 15

### Methyl 3-[4-(2-{4-chloro-5-[(Z)-(methoxyimino)(phenyl)methyl]-1H-pyrrolo[2,3-b]pyridin-1-yl}ethoxy)phenyl]-2-(2,2,2-trifluoroethoxy)-propanoate

### Step A: 4-Chloro-1-(triisopropylsilyl)-1H-pyrrolo[2,3-b]pyridine

In a round-bottomed flask under argon, 6 g (39.32 mmol) of 4-chloro-1H-pyrrolo[2,3-b]pyridinc are dissolved in 90 ml of tetrahydrofuran and cooled to 0° C. 1.73 g (43.25 mmol) of sodium hydride (60% in oil) are then added in portions. The mixture is stirred for 15 minutes at 0° C. 8.42 ml (39.32 mmol) of triisopropylsilyl chloride are then added. The reaction mixture is stirred for 3 hours at reflux. It is hydrolysed with 50 ml of saturated ammonium chloride solution and extracted 3 times with 50 ml of dichloromethane. The organic phases are dried over magnesium sulphate, filtered and evaporated under reduced pressure.

The residue is purified by chromatography on silica gel (pure petroleum ether, then petroleum ether/ethyl acetate: 95/5) to yield the title product in the form of a yellowish oil.

MS: m/z=309 [M+H]⁺³⁵Cl; 311 [M+H]⁺³⁷Cl.

### Step B: [4-Chloro-1-(triisopropylsilyl)-1H-pyrrolo[2,3-b]pyridin-5-yl](phenyl)methanol

In a three-necked flask under argon, 1.5 g (4.86 mmol) of the compound obtained in Step A are dissolved in 50 ml of anhydrous tetrahydrofuran and cooled to −78° C. 8.2 ml (10.69 mmol) of sec-butyllithium (1.3M in solution in a hexane/cyclohexane mixture) are then added dropwise. The reaction mixture is stirred for 30 minutes a −78° C. 1.3 ml (12.15 mmol) of distilled benzaldehyde are then added. The mixture is stirred for 14 hours while returning to ambient temperature. It is hydrolysed with 50 ml of saturated ammonium chloride solution and extracted with dichloromethane (3 times, 50 ml each time). The organic phases are dried over magnesium sulphate, filtered and evaporated under reduced pressure.

The residue is purified by chromatography on silica gel (petroleum ether/ethyl acetate: 98/2) to yield the title product in the form of a colourless oil.

MS: m/z=416 [M+H]⁺³⁵Cl; 418 [M+H]⁺³⁷Cl.

### Step C: [4-Chloro-1-(triisopropylsilyl)-1H-pyrrolo[2,3-b]pyridin-5-yl](phenyl)methanone

The compound obtained in Step B (1.4 g) is dissolved in 90 ml of toluene and 2.93 g (33.70 mmol) of manganese dioxide are added. A Dean Stark apparatus is put in position. The reaction mixture is stirred for 18 hours at reflux and then allowed to return to ambient temperature. The mixture is then filtered over Celite and the filtrate is evaporated. The residue is purified by chromatography on silica gel (petroleum ether/ethyl acetate: 95/5) to obtain the title compound in the form of a white solid.

Melting point: 104° C.

MS: m/z=413 [M+H]⁺³⁵Cl; 415 [M+H]⁺³⁷Cl.

### Step D: (Z)-[4-Chloro-1-(triisopropylsilyl)-1H-pyrrolo[2,3-b]pyridin-5-yl](phenyl)methanone O-methyloxime

In a round-bottomed flask under argon, 2.3 g (5.57 mmol-1 eq.) of the compound obtained in Step C are dissolved in 90 ml of absolute ethanol. 0.68 ml (8.36 mmol) of pyridine and 0.698 g (8.36 mmol) of O-methylhydroxylamine hydrochloride are then added. The reaction mixture is stirred for 46 hours at reflux. The solvent is then evaporated off. The residue is taken up in 100 ml of water and extracted 3 times with 50 ml of dichloromethane. The combined organic phases are dried over magnesium sulphate, filtered and evaporated under reduced pressure. The crude product is purified and the 2 oximes separated on a BIOTAGE column in 250 mg fractions (petroleum ether/ethyl acetate:500 ml 8/2, 150 ml 8/2 to 7/3 and 400 ml 7/3).

Z isomer: white solid.

Melting point: 110° C.

### Step E: (Z)-(4-Chloro-1H-pyrrolo[2,3-b]pyridin-5-yl) (phenyl)methanone O-methyloxime

In a round-bottomed flask under an argon atmosphere, 0.800 g (1.81 mmol) of the compound obtained in Step D is dissolved in 8 ml of tetrahydrofuran. 4 ml (3.98 mmol) of tetrabutylammonium fluoride (1M in solution in tetrahydrofuran) are then added. The reaction mixture is stirred for 19 hours at ambient temperature. It is then hydrolysed with 10 ml of water and extracted 3 times with 10 ml of dichloromethane. The organic phases are dried over magnesium sulphate, filtered and evaporated under reduced pressure. The residue obtained is purified by chromatography on silica gel (petroleum ether/ethyl acetate:8/2, 7/3 then 6/4) to yield the title product in the form of a white solid.

Melting point: 176° C.

MS: m/z=286 [M+H]⁺³⁵C; 288 [M+H]⁺³⁷Cl.

### Step F: Methyl 3-[4-(2-{4-chloro-5-[(Z)-(methoxyimino)(phenyl)methyl]-1H-pyrrolo[2,3-b]pyridin-1-yl}ethoxy)phenyl]-2-(2,2,2-trifluoroethoxy)-propanoate

In a two-necked flask under an argon atmosphere, the compound obtained in Step E (0.300 g) is dissolved in 5 ml of anhydrous N,N-dimethylformamide, then 0.063 g (1.58 mmol) of sodium hydride (60% in oil) is added. The mixture is stirred for 1 hour and 30 minutes at ambient temperature and cooled to 0° C. The anion obtained is then added to a mixture containing 0.485 g (1.26 mmol) of methyl 3-[4-(2-bromoethoxy)phenyl]-2-(2,2,2-trifluoroethoxy)propanoate, 17 mg of potassium iodide and 8 ml of anhydrous N,N-dimethylformamide. The reaction mixture is stirred for 2 hours at ambient temperature.

The solvent is then evaporated off and the residue is taken up in 10 ml of water. It is extracted 3 times with 10 ml of ethyl acetate. The combined organic phases are washed with 20 ml of saturated sodium chloride solution, and then dried over magnesium sulphate, filtered and evaporated under reduced pressure. The crude product is purified by chromatography on silica gel (petroleum ether/ethyl acetate: 8/2, 7/3 then 6/4) to yield the title product in the form of a colourless oil.

MS: m/z=590 [M+H]⁺³⁵C; 592 [M+H]⁺³⁷Cl.

### EXAMPLE 16

### 3-[4-(2-{4-Chloro-5-[(Z)-(methoxyimino)(phenyl)methyl]-1H-pyrrolo[2,3-b]pyridin-1-yl}ethoxy)phenyl]-2-(2,2,2-trifluoro-ethoxy)propanoic acid

The compound obtained in Example 15 (0.300 g) is dissolved in tetrahydrofuran and cooled to 0° C. 0.064 g (1.53 mmol) of lithium hydroxide monohydrate dissolved in 10 ml of water is then added. The reaction mixture is stirred for an hour and a half from 0° C. to ambient temperature. The solvent is evaporated off and the residue is dissolved in 10 ml of water. After acidification using aqueous acetic acid, it is extracted 3 times with 10 ml of dichloromethane. The organic phases are dried over magnesium sulphate, filtered and evaporated. The white powder obtained is washed with hexane, filtered through a glass frit and dried in vacuo.

Melting point: 186° C.

MS: m/z=576 [M+H]⁺³⁵Cl; 578 [M+H]⁺³⁷Cl.

### EXAMPLE 17

### Methyl 3-[4-(2-{6-[cyclopropyl(methoxyimino)methyl]-1H-pyrrolo[2,3-b]pyridin-1-yl}ethoxy)phenyl]-2-ethoxypropanoate

The procedure is as in Step B of Example 1, starting from the compound obtained in Preparation 1.

### EXAMPLE 18

### Methyl 3-[4-(2-{6-[cyclopropyl(methoxyimino)methyl]-1H-pyrrolo[2,3-b]pyridin-1-yl}ethoxy)phenyl]-2-(2,2,2-trifluoro-ethoxy)propanoate

The procedure is as in Step B of Example 1, starting from the compound obtained in Preparation 2.

### EXAMPLE 19

### Methyl 3-[4-(2-{6-[cyclohexyl(methoxyimino)methyl]-1H-pyrrolo[2,3-b]pyridin-1-yl}ethoxy)phenyl]-2-ethoxypropanoate

The procedure is as in Step B of Example 1, starting from the compound obtained in Preparation 3.

### EXAMPLE 20

### Methyl (2S)-3-[4-(2-{6-[cyclohexyl(methoxyimino)methyl]-1H-pyrrolo[2,3-b]pyridin-1-yl}ethoxy)phenyl]-2-ethoxypropanoate

The procedure is as in Step B of Example 1, starting from the compound obtained in Preparation 4.

### EXAMPLE 21

### Methyl 3-[4-(2-{6-[cyclohexyl(methoxyimino)methyl]-1H-pyrrolo[2,3-b]pyridin-1-yl}ethoxy)phenyl]-2-(2,2,2-trifluoroethoxy)propanoate

The procedure is as in Step B of Example 1, starting from the compound obtained in Preparation 5.

### EXAMPLE 22

### Methyl (2S)-3-[4-(2-{6-[cyclohexyl(methoxyimino)methyl]-1H-pyrrolo[2,3-b]pyridin-1-yl}ethoxy)phenyl]-2-(2,2,2-trifluoro-ethoxy)propanoate

The procedure is as in Step B of Example 1, starting from the compound obtained in Preparation 6.

### EXAMPLE 23

### 3-[4-(2-{6-[Cyclopropyl(methoxyimino)methyl]-1H-pyrrolo[2,3-b]pyridin-1-yl}ethoxy)phenyl]-2-ethoxypropanoic acid

The procedure is as in Step B of Example 1, starting from the compound obtained in Preparation 7.

### EXAMPLE 24

### 3-[4-(2-{6-[Cyclopropyl(methoxyimino)methyl]-1H-pyrrolo[2,3-b]pyridin-1-yl}ethoxy)phenyl]-2-(2,2,2-trifluoroethoxy)propanoic acid

The procedure is as in Step B of Example 1, starting from the compound obtained in Preparation 8.

### EXAMPLE 25

### 3-[4-(2-{6-[Cyclohexyl(methoxyimino)methyl]-1H-pyrrolo[2,3-b]pyridin-1-yl}ethoxy)phenyl]-2-ethoxypropanoic acid

The procedure is as in Step B of Example 1, starting from the compound obtained in Preparation 9.

### EXAMPLE 26

### (2S)-3-[4-(2-{6-[Cyclohexyl(methoxyimino)methyl]-1H-pyrrolo[2,3-b]pyridin-1-yl}ethoxy)phenyl]-2-ethoxypropanoic acid

The procedure is as in Step B of Example 1, starting from the compound obtained in Preparation 10.

### EXAMPLE 27

### 3-[4-(2-{6-[Cyclohexyl(methoxyimino)methyl]-1H-pyrrolo[2,3-b]pyridin-1-yl}ethoxy)phenyl]-2-(2,2,2-trifluoroethoxy)propanoic acid

The procedure is as in Step B of Example 1, starting from the compound obtained in Preparation 11.

### EXAMPLE 28

### (2S)-3-[4-(2-{6-[Cyclohexyl(methoxyimino)methyl]-1H-pyrrolo[2,3-b]pyridin-1-yl}ethoxy)phenyl]-2-(2,2,2-trifluoroethoxy)propanoic acid

The procedure is as in Step B of Example 1, starting from the compound obtained in Preparation 12.

### EXAMPLE 29

### Methyl 2-ethoxy-3-[4-(2-{6-[(methoxyimino)(4-pyridinyl)methyl]-1H-pyrrolo[2,3-b]pyridin-1-yl}ethoxy)phenyl]propanoate

The procedure is as in Steps B and C of Example 1, starting from 4-pyridinyl(1H-pyrrolo[2,3-b]pyridin-6-yl)methanone.

### EXAMPLE 30

### 2-Ethoxy-3-[4-(2-{6-[(methoxyimino)(4-pyridinyl)methyl]-1H-pyrrolo[2,3-b]pyridin-1-yl}ethoxy)phenyl]propanoic-acid

The procedure is as in Example 5, starting from the compound obtained in Example 29.

### EXAMPLE 31

### Methyl (2S)-3-[4-(2-{6-[(methoxyimino)(phenyl)methyl]-1H-pyrrolo[2,3-b]pyridin-1-yl}ethoxy)phenyl]-2-(2,2,2-trifluoroethoxy)-propanoate

The procedure is as in Example 1, without separation of the isomers obtained in Step B and with the replacement of methyl 3-[4-(2-bromoethoxy)phenyl]-2-ethoxypropanoate with methyl (2S)-3-[4-(2-bromoethoxy)phenyl]-2-(2,2,2-trifluoroethoxy)propanoate in Step C.

### EXAMPLE 32

### (2S)-3-[4-(2-{6-[(Methoxyimino) (phenyl)methyl]-1H-pyrrolo[2,3-b]pyridin-1-yl}ethoxy)phenyl]-2-(2,2,2-trifluoroethoxy)propanoic acid

The procedure is as in Example 5, starting from the compound obtained in Example 31.

### EXAMPLE 33

### Methyl 2-ethoxy-3-[4-(2-{6-[(phenoxyimino)(phenyl)methyl]-1H-pyrrolo[2,3-b]pyridin-1-yl}ethoxy)phenyl]propanoate

The procedure is as in Example 1, with the replacement of methoxyammonium chloride with phenoxyammonium chloride in Step B and without separation of the isomers obtained.

### EXAMPLE 34

### 2-Ethoxy-3-[4-(2-{6-[(phenoxyimino) (phenyl)methyl]-1H-pyrrolo[2,3-b]pyridin-1-yl}ethoxy)phenyl]propanoic acid

The procedure is as in Example 5, starting from the compound obtained in Example 33.

### EXAMPLE 35

### Methyl 3-[4-(2-{6-[cyclopentyl(methoxyimino)methyl]-1H-pyrrolo[2,3-b]pyridin-1-yl}ethoxy)phenyl]-2-ethoxypropanoate

The procedure is as in Step B of Example 1, starting from the compound obtained in Preparation 13.

### EXAMPLE 36

### 3-[4-(2-{6-[Cyclopentyl(methoxyimino)methyl]-1H-pyrrolo[2,3-b]pyridin-1-yl}ethoxy)phenyl]-2-ethoxypropanoic acid

The procedure is as in Step B of Example 1, starting from the compound obtained in Preparation 14.

### EXAMPLE 37

### Methyl 2-ethoxy-3-[4-(2-{6-[(methoxyimino)(phenyl)methyl]-1H-pyrrolo[2,3-b]pyridin-1-yl}ethoxy)phenyl]propanoate

The procedure is as in Example 1, without separation of the isomers obtained in Step B.

### EXAMPLE 38

### 2-Ethoxy-3-[4-(2-{6-[(methoxyimino)(phenyl)methyl]-1H-pyrrolo[2,3-b]pyridin-1-yl}ethoxy)phenyl]propanoic acid

The procedure is as in Example 5, starting from the compound obtained in Example 37.

### EXAMPLE 39

### 3-[4-(2-{6-[Cyclopropyl(hydroxyimino)methyl]-1H-pyrrolo[2,3-b]pyridin-1-yl}ethoxy)phenyl]-2-(2,2,2-trifluoroethoxy)propanoic acid

Add the compound obtained in Preparation 8 (0.00198 mol), hydroxylamine hydrochloride (0.00594 mol) and pyridine (0.00594 mol) to 30 ml of methanol. Heat at reflux for 3 hours. Evaporate to dryness. Add 100 ml of hydrochloric acid (IN) and then extract with 50 ml of dichloromethane twice, and evaporate under reduced pressure to yield the title product in the form of a white solid.

### EXAMPLE 40

### 2-Ethoxy-3-[4-(2-{6-[(hydroxyimino)(phenyl)methyl]-1H-pyrrolo[2,3-b]pyridin-1-yl}ethoxy)phenyl]propanoic acid

The procedure is as in Example 1, with the replacement of methoxyammonium chloride with hydroxylamine hydrochloride in Step B.

Yellowish oil.

### EXAMPLE 41

### 3-[4-(2-{6-[Cyclopropyl(hydroxyimino)methyl]-1H-pyrrolo[2,3-b]pyridin-1-yl}ethoxy)phenyl]-2-ethoxypropanoic acid

The procedure is as in Example 39, starting from the compound obtained in Preparation 7.

Yellowish solid.

MS: m/z=438.5 [M+H]⁺.

### Pharmacological Study

### EXAMPLE A

### Acute Toxicity Study

The acute toxicity was evaluated after oral administration to groups each comprising 8 mice (26±2 grams). The animals were observed at regular intervals during the course of the first day, and daily for two weeks following the treatment. The LD₅₀ (dose that causes the death of 50% of the animals) was evaluated and demonstrated the low toxicity of the compounds of the invention.

### EXAMPLE B

### Effectiveness in Genetic Models

Mutations in laboratory animals and also different sensitivities to dietary regimens have allowed the development of animal models having non-insulin-dependent diabetes and hyperlipidaemia associated with obesity and with resistance to insulin. Genetic mice models (ob/ob) (Diabetes, 1982, 31 (1), 1-6) and Zucker (fa/fa) rats have been developed by various laboratories in order to understand the physiopathology of those diseases and test the effectiveness of new antidiabetic compounds (Diabetes, 1983, 32, 830-838).

Antidiabetic and Hypolipaemic Effect in the ob/ob Mouse

The 10-week-old male ob/ob mouse (Harlan) is used for the in vivo tests. The animals are kept in a light-darkness cycle of 12 hours at 25° C. This mouse has a basal hyperglycaemia of 2 g/l. The animals are randomly selected with regard to their glycaemia to form groups of eight. The compounds tested by the oral route are dissolved in a mixture of hydroxyethyl cellulose (HEC 1%) to be administered at 3 mg/kg in a volume of 10 ml/kg once per day for four days. The control group receives the solvents under the same conditions as the treated groups. The activity of the products is evaluated by measuring glycaemia, triglyceridaemia and insulinaemia 24 hours after the final administration and by measuring body weight daily.

The compounds of the invention demonstrate a very good capacity to lower glycaemia that is comparable to the effects obtained with rosiglitazone, which is used as reference substance.

By way of example, administered at a dose of 3 mg/kg, the compound of Example 32 exhibits a 37% reduction in triglyceridaemia compared with the control group, a 23% reduction in insulinaemia compared with the control group and a 43% reduction in glycaemia compared with the control.

Furthermore, no side effect was observed during the in vivo tests.

### EXAMPLE C: Pharmaceutical composition

| | |
|---|---|
| 1000 tablets each containing a dose of 5 mg of | 5 g |
| (2S)-3-[4-(2-{6-[(methoxyimino)(phenyl)- | |
| methyl]-1H-pyrrolo[2,3-b]pyridin-1- | |
| yl}ethoxy)phenyl]-2-(2,2,2-trifluoroethoxy)propanoic acid | |
| (Example 32) | |
| wheat starch | 20 g |
| maize starch | 20 g |
| lactose | 30 g |
| magnesium stearate | 2 g |
| silica | 1 g |
| hydroxypropyl cellulose | 2 g |

### EXAMPLE D

### Variation in Body Weight

Male C57 Black 6 ob/ob mice 8 to 12 weeks old were used. After being placed in quarantine for one week, they were weighed and then randomly selected as a function of their weight, and 6 homogeneous groups (starting weight not significantly different) were formed. After weighing the animals, the different associations to be tested are injected intraperitoneally once per day for 7 days. The molecules are injected in a 5% DMSO/15% Solutol/q.s. H₂O solution heated to 65° C. to ensure good dissolution. The solution is in addition preheated prior to injection. The mice are weighed every day and the weight attained after 7 days of treatment is recorded.

The results obtained clearly demonstrate
that the association according to the invention between a compound of formula (I) or (V) and an antioxidant agent allows a significant reduction in the weight of the obese mice to be achieved,
that there is a synergy between the 2 components of the association, the loss in weight ascertained being far greater using the association than when using each component administered on its own.

### EXAMPLE E

### Pharmaceutical Composition

| | |
|---|---|
| 100 tablets each containing a dose of 30 mg of | 3 g |
| (2S)-3-[4-(2-{6-[(methoxyimino)(phenyl)- | |
| methyl]-1H-pyrrolo[2,3-b]pyridin-1- | |
| yl}ethoxy)phenyl]-2-(2,2,2-trifluoroethoxy)propanoic | |
| acid (Example 32) and 10 mg of coenzyme Q₁₀ | |
| (2S)-3-[4-(2-{6-[(methoxyimino)(phenyl)methyl]-1H- | |
| pyrrolo[2,3-b]pyridin-1-yl}ethoxy)phenyl]-2- | |
| (2,2,2-trifluoroethoxy)propanoic acid (Example 32) | |
| coenzyme Q₁₀ | 1 g |
| wheat starch | 20 g |
| maize starch | 20 g |
| lactose | 30 g |
| magnesium stearate | 2 g |
| silica | 1 g |
| hydroxypropyl cellulose | 2 g |

1-29. (canceled)

30. A compound selected from those of formula (I): 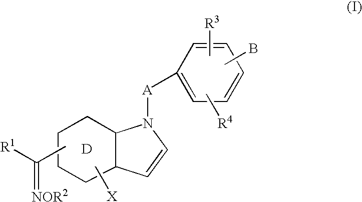
wherein:
R¹ represents an aryl, heteroaryl or (C₃-C₈)cycloalkyl group,
R² represents a hydrogen atom, or a linear or branched (C₁-C₆)alkyl group, an aryl group or an aryl-(C₁-C₆)alkyl group in which the alkyl moiety may be linear or branched,
X represents a hydrogen or halogen atom or a linear or branched (C₁-C₆)alkyl group,
R³ and R⁴, which may be identical or different, each represent a hydrogen or halogen atom, a linear or branched (C₁-C₆)alkyl, linear or branched (C₁-C₆)alkoxy or linear or branched (C₁-C₆)alkylamino group or a di(C₁-C₆)alkylamino group in which the alkyl moieties may be linear or branched,
D represents a pyridine nucleus,
A represents a (C₁-C₆)alkylene chain in which a CH₂ group may be replaced by a hetero atom selected from oxygen and sulphur, or by an NRₐ group (wherein Rₐ represents a hydrogen atom or a linear or branched (C₁-C₆)alkyl group),
B represents a linear or branched (C₁-C₆)alkyl group or a linear or branched (C₂-C₆)alkenyl group, those groups being substituted by a group of formula (II): 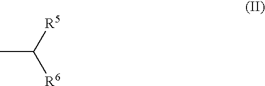
wherein R⁵ represents a —COOR group and R⁶ represents an —OR′ group wherein R and R′, which may be identical or different, each represent a hydrogen atom, or a linear or branched (C₁-C₆)alkyl group unsubstituted or substituted by one or more halogen atoms,
it being understood that:
the oxime R¹—C(═N—OR²)— may have the Z or E configuration,
aryl means a phenyl, naphthyl or biphenyl group, which groups may be partially hydrogenated,
heteroaryl means an aromatic mono- or bi-cyclic group containing from 5 to 10 ring members, wherein the bicyclic heteroaryl groups may be partially hydrogenated on one of the rings, and wherein each ring contains from 1 to 3 hetero atoms selected from oxygen, nitrogen and sulphur,
wherein the aryl and heteroaryl groups may be substituted by from 1 to 3 groups selected from linear or branched (C₁-C₆)alkyl, linear or branched (C₁-C₆)-polyhaloalkyl, linear or branched (C₁-C₆)alkoxy, hydroxy, carboxy, formyl, NR (wherein R_{b} and R_{c}, which may be identical or different, each represent a hydrogen atom, a linear or branched (C₁-C₆)alkyl group, an aryl group or a heteroaryl group), ester, amido, nitro, cyano, and halogen atoms,
its enantiomers and diastereoisomers, and pharmaceutically acceptable addition salts thereof with an acid or a base.

31. The compound of claim 30, wherein R¹ represents a phenyl group.

32. The compound of claim 30, wherein R² represents an alkyl group.

33. The compound of claim 30, wherein A represents an ethyleneoxy group.

34. The compound of claim 30, wherein D with the ring to which it is fused represents a 1H-pyrrolo[2,3-b]pyridine system.

35. The compound of claim 30, wherein X represents a hydrogen atom.

36. The compound of claim 30, wherein R³ and R⁴ represent a hydrogen atom.

37. The compound of claim 30, wherein B represents a group —CH₂—CH(R⁵)(R⁶).

38. The compound of claim 30, which is selected from:
methyl 2-ethoxy-3-[4-(2-{6-[(E)-(methoxyimi no)(phenyl)methyl]-1H-pyrrolo[2,3-b]pyridin-1-yl}ethoxy)phenyl]propanoate,
methyl (2S)-2-ethoxy-3-[4-(2-{6-[(E)-(methoxyimino)(phenyl)methyl]-1H-pyrrolo[2,3-b]pyridin-1-yl}ethoxy)phenyl]propanoate,
methyl 3-[4-(2-{6-[(E)-(methoxyimino)(phenyl)methyl]-1H-pyrrolo[2,3-b]pyridin-1-yl}ethoxy)phenyl]-2-(2,2,2-trifluoroethoxy)propanoate,
methyl (2S)-3-[4-(2-{6-[(E)-(methoxyimino)(phenyl)methyl]-1H-pyrrolo[2,3-b]pyridin-1-yl}ethoxy)phenyl]-2-(2,2,2-trifluoroethoxy)propanoate,
2-ethoxy-3-[4-(2-{6-[(E)-(methoxyimino)(phenyl)methyl]-1H-pyrrolo[2,3-b]pyridin-1-yl}ethoxy)phenyl]propanoic acid,
(2S)-2-ethoxy-3-[4-(2-{6-[(E)-(methoxyimino)(phenyl)methyl]-1H-pyrrolo[2,3-b]pyridin-1-yl}ethoxy)phenyl]propanoic acid,
3-[4-(2-{6-[(E)-(methoxyimino)(phenyl)methyl]-1H-pyrrolo[2,3-b]pyridin-1-yl}ethoxy)phenyl]-2-(2,2,2-trifluoroethoxy)propanoic acid,
(2S)-3-[4-(2-{6-[(E)-(methoxyimino)(phenyl)methyl]-1H-pyrrolo[2,3-b]pyridin-1-yl}ethoxy)phenyl]-2-(2,2,2-trifluoroethoxy)propanoic acid,
methyl 2-ethoxy-3-[4-(2-{6-[(Z)-(methoxyimino) (phenyl)methyl]-1H-pyrrolo[2,3-b]pyridin-1-yl}ethoxy)phenyl]propanoate,
2-ethoxy-3-[4-(2-{6-[(Z)-(methoxyimino)(phenyl)methyl]-1H-pyrrolo[2,3-b]pyridin-1-yl}ethoxy)phenyl]propanoic acid,
methyl 2-ethoxy-3-[4-(2-{5-[(methoxyimino)(phenyl)methyl]-1H-pyrrolo[2,3-b]pyridin-1-yl}ethoxy)phenyl]propanoate,
methyl 3-[4-(2-{5-[(methoxyimino)(phenyl)methyl]-1H-pyrrolo[2,3-b]pyridin-1-yl}ethoxy)phenyl]-2-(2,2,2-trifluoroethoxy)propanoate,
2-ethoxy-3-[4-(2-{5-[(methoxyimino)(phenyl)methyl]-1H-pyrrolo[2,3-b]pyridin-1-yl}ethoxy)phenyl]propanoic acid,
3-[4-(2-{5-[(methoxyimino)(phenyl)methyl]-1H-pyrrolo[2,3-b]pyridin-1-yl}ethoxy)phenyl]-2-(2,2,2-trifluoroethoxy)propanoic acid,
methyl 3-[4-(2-{4-chloro-5-[(Z)-(methoxyimino)(phenyl)methyl]-1H-pyrrolo[2,3-b]pyridin-1-yl}ethoxy)phenyl]-2-(2,2,2-trifluoroethoxy)propanoate,
3-[4-(2-{4-chloro-5-[(Z)-(methoxyimino) (phenyl)methyl]-1H-pyrrolo[2,3-b]pyridin-1-yl}ethoxy)phenyl]-2-(2,2,2-trifluoroethoxy)propanoic acid,
methyl 3-[4-(2-{6-[cyclopropyl(methoxyimino)methyl]-1H-pyrrolo[2,3-b]pyridin-1-yl}ethoxy)phenyl]-2-ethoxypropanoate,
methyl 3-[4-(2-{6-[cyclopropyl(methoxyimino)methyl]-1H-pyrrolo[2,3-b]pyridin-1-yl}ethoxy)phenyl]-2-(2,2,2-trifluoroethoxy)propanoate,
methyl 3-[4-(2-{6-[cyclohexyl(methoxyimino)methyl]-1H-pyrrolo[2,3-b]pyridin-1-yl}ethoxy)phenyl]-2-ethoxypropanoate,
methyl (2S)-3-[4-(2-{6-[cyclohexyl(methoxyimino)methyl]-1H-pyrrolo[2,3-b]pyridin-1-yl}ethoxy)phenyl]-2-ethoxypropanoate,
methyl 3-[4-(2-{6-[cyclohexyl(methoxyimino)methyl]-1H-pyrrolo[2,3-b]pyridin-1-yl}ethoxy)phenyl]-2-(2,2,2-trifluoroethoxy)propanoate,
methyl (2S)-3-[4-(2-{6-[cyclohexyl(methoxyimino)methyl]-1H-pyrrolo[2,3-b]pyridin-1-yl}ethoxy)phenyl]-2-(2,2,2-trifluoroethoxy)propanoate,
3-[4-(2-{6-[cyclopropyl(methoxyimino)methyl]-1H-pyrrolo[2,3-b]pyridin-1-yl}ethoxy)phenyl]-2-ethoxypropanoic acid,
3-[4-(2-{6-[cyclopropyl(methoxyimino)methyl]-1H-pyrrolo[2,3-b]pyridin-1-yl}ethoxy)phenyl]-2-(2,2,2-trifluoroethoxy)propanoic acid,
3-[4-(2-{6-[cyclohexyl(methoxyimino)methyl]-1H-pyrrolo[2,3-b]pyridin-1-yl}ethoxy)phenyl]-2-ethoxypropanoic acid,
(2S)-3-[4-(2-{6-[cyclohexyl(methoxyimino)methyl]-1H-pyrrolo[2,3-b]pyridin-1-yl}-ethoxy)phenyl]-2-ethoxypropanoic acid,
3-[4-(2-{6-[cyclohexyl(methoxyimino)methyl]-1H-pyrrolo[2,3-b]pyridin-1-yl}ethoxy)phenyl]-2-(2,2,2-trifluoroethoxy)propanoic acid,
(2S)-3-[4-(2-{6-[cyclohexyl(methoxyimino)methyl]-1H-pyrrolo[2,3-b]pyridin-1-yl}ethoxy)phenyl]-2-(2,2,2-trifluoroethoxy)propanoic acid,
methyl 2-ethoxy-3-[4-(2-{6-[(methoxyimino)(4-pyridinyl)methyl]-1H-pyrrolo[2,3-b]pyridin-1-yl}ethoxy)phenyl]propanoate,
2-ethoxy-3-[4-(2-{6-[(methoxyimino)(4-pyridinyl)methyl]-1H-pyrrolo[2,3-b]pyridin-1-yl}ethoxy)phenyl]propanoic acid,
methyl (2S)-3-[4-(2-{6-[(methoxyimino)(phenyl)methyl]-1H-pyrrolo[2,3-b]pyridin-1-yl}ethoxy)phenyl]-2-(2,2,2-trifluoroethoxy)propanoate,
(2S)-3-[4-(2-{6-[(methoxyimino)(phenyl)methyl]-1H-pyrrolo[2,3-b]pyridin-1-yl}ethoxy)phenyl]-2-(2,2,2-trifluoroethoxy)propanoic acid,
methyl 2-ethoxy-3-[4-(2-{6-[(phenoxyimino) (phenyl)methyl]-1H-pyrrolo[2,3-b]pyridin-1-yl}ethoxy)phenyl]propanoate,
2-ethoxy-3-[4-(2-{6-[(phenoxyimino)(phenyl)methyl]-1H-pyrrolo[2,3-b]pyridin-1-yl}ethoxy)phenyl]propanoic acid,
methyl 3-[4-(2-{6-[cyclopentyl(methoxyimino)methyl]-1H-pyrrolo[2,3-b]pyridin-1-yl}ethoxy)phenyl]-2-ethoxypropanoate,
3-[4-(2-{6-[cyclopentyl(methoxyimino)methyl]-1H-pyrrolo[2,3-b]pyridin-1-yl}ethoxy)phenyl]-2-ethoxypropanoic acid,
methyl 2-ethoxy-3-[4-(2-{6-[(methoxyimino)(phenyl)methyl]-1H-pyrrolo[2,3-b]pyridin-1-yl}ethoxy)phenyl]propanoate,
2-ethoxy-3-[4-(2-{6-[(methoxyimino)(phenyl)methyl]-1H-pyrrolo[2,3-b]pyridin-1-yl}ethoxy)phenyl]propanoic acid,
3-[4-(2-{6-[cyclopropyl(hydroxyimino)methyl]-1H-pyrrolo[2,3-b]pyridin-1-yl}ethoxy)phenyl]-2-(2,2,2-trifluoroethoxy)propanoic acid,
2-ethoxy-3-[4-(2-{6-[(hydroxyimino)(phenyl)methyl]-1H-pyrrolo[2,3-b]pyridin-1-yl}ethoxy)phenyl]propanoic acid,
3-[4-(2-{6-[cyclopropyl(hydroxyimino)methyl]-1H-pyrrolo[2,3-b]pyridin-1-yl}ethoxy)phenyl]-2-ethoxypropanoic acid.

39. A process for the preparation of a compound of claim 30, wherein a compound of formula (III): 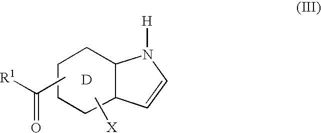
is used as a starting material,
wherein D, R¹ and X are as defined for formula (I),
which compound is condensed in basic medium with a compound of formula (IV): 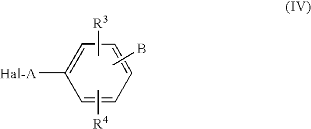
wherein A, B, R³ and R⁴ are as defined for formula (I) and Hal represents a halogen atom, to yield a compound of formula (V): 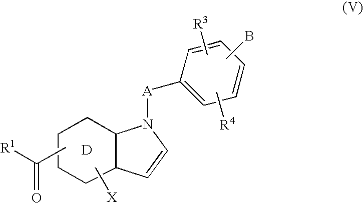
wherein R¹, R³, R⁴, A, B, D and X are as defined for formula (I),
which compound is subjected to the action of a compound of formula R²⁰—NH₂
wherein R² is as defined for formula (I) to yield a compound of formula (I): 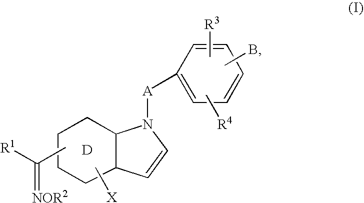
which compound may be purified according to a conventional separation technique, is converted, if desired, into addition salts with a pharmaceutically acceptable acid or base, and is optionally separated into isomers according to a conventional separation technique.

40. A process for the preparation of a compound of claim 30, wherein a compound of formula (III): 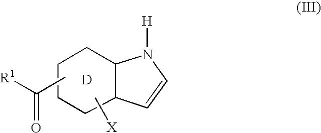
is used as starting material,
wherein D, R¹ and X are as defined for formula (I),
which compound is condensed with a compound of formula R²⁰—NH₂ wherein R² is as defined for formula (I) to yield a compound of formula (VI): 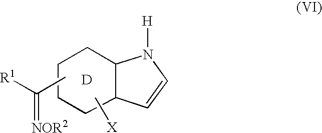
wherein R¹, R², D and X are as defined for formula (I), which compound is condensed in basic medium with a compound of formula (IV): 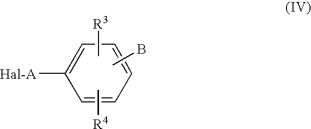
wherein A, B, R³ and R⁴ are as defined for formula (I) and Hal represents a halogen atom to yield a compound of formula (I): 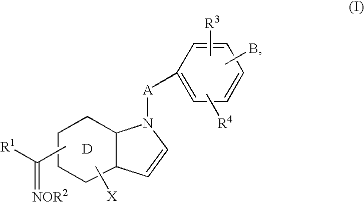
which compound may be purified according to a conventional separation technique, is converted, if desired, into addition salts with a pharmaceutically acceptable acid or base, and is optionally separated into isomers according to a conventional separation technique.

41. A compound selected from those of formula (V): 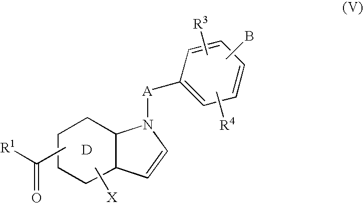
wherein:
R¹ represents an aryl, heteroaryl or (C₃-C₈)cycloalkyl group,
X represents a hydrogen or halogen atom or a linear or branched (C₁-C₆)alkyl group,
R³ and R⁴, which may be identical or different, each represent a hydrogen or halogen atom, a linear or branched (C₁-C₆)alkyl, linear or branched (C₁-C₆)alkoxy or linear or branched (C₁-C₆)alkylamino group or a di(C₁-C₆)alkylamino group in which the alkyl moieties may be linear or branched,
D represents a pyridine nucleus,
A represents a (C₁-C₆)alkylene chain in which a CH₂ group may be replaced by a hetero atom selected from oxygen and sulphur, or by an NRₐ group (wherein Rₐ represents a hydrogen atom or a linear or branched (C₁-C₆)alkyl group),
B represents a linear or branched (C₁-C₆)alkyl group or a linear or branched (C₂-C₆)alkenyl group, those groups being substituted by a group of formula (II): 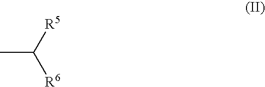
wherein R⁵ represents a —COOR group and R⁶ represents an —OR′ group wherein R and R′, which may be identical or different, each represent a hydrogen atom, or a linear or branched (C₁-C₆)alkyl group unsubstituted or substituted by one or more halogen atoms,
it being understood that:
the oxime R¹—C(═N—OR²)— may have the Z or E configuration,
aryl means a phenyl, naphthyl or biphenyl group, which groups may be partially hydrogenated,
heteroaryl means an aromatic mono- or bi-cyclic group containing from 5 to 10 ring members, wherein the bicyclic heteroaryl groups may be partially hydrogenated on one of the rings, and wherein each ring contains from 1 to 3 hetero atoms selected from oxygen, nitrogen and sulphur,
wherein the aryl and heteroaryl groups may be substituted by from 1 to 3 groups selected from linear or branched (C₁-C₆)alkyl, linear or branched (C₁-C₆)-polyhaloalkyl, linear or branched (C₁-C₆)alkoxy, hydroxy, carboxy, formyl, NR_{d}R_{c} (wherein R_{b} and R_{c}, which may be identical or different, each represent a hydrogen atom, a linear or branched (C₁-C₆)alkyl group, an aryl group or a heteroaryl group), ester, amido, nitro, cyano, and halogen atoms,
for use as an intermediate in the synthesis of compounds of formula (I).

42. A compound of claim 41, which is selected from:
methyl 3-(4-{2-[6-(cyclopropylcarbonyl)-1H-pyrrolo[2,3-b]pyridin-1-yl]ethoxy}phenyl)-2-ethoxypropanoate,
methyl 3-(4-{2-[6-(cyclopropylcarbonyl)-1H-pyrrolo[2,3-b]pyridin-1-yl]ethoxy}phenyl)-2-(2,2,2-trifluoroethoxy)propanoate,
methyl 3-(4-{2-[6-(cyclohexylcarbonyl)-1H-pyrrolo[2,3-b]pyridin-1-yl]ethoxy}phenyl)-2-ethoxypropanoate,
methyl(2S)-3-(4-{2-[6-(cyclohexylcarbonyl)-1H-pyrrolo[2,3-b]pyridin-1-yl]ethoxy}phenyl)-2-ethoxypropanoate,
methyl 3-(4-{2-[6-(cyclohexylcarbonyl)-1H-pyrrolo[2,3-b]pyridin-1-yl]ethoxy}phenyl)-2-(2,2,2-trifluoroethoxy)propanoate,
methyl (2S)-3-(4-{2-[6-(cyclohexylcarbonyl)-1H-pyrrolo[2,3-b]pyridin-1-yl]ethoxy}phenyl)-2-(2,2,2-trifluoroethoxy)propanoate,
3-(4-{2-[6-(cyclopropylcarbonyl)-1H-pyrrolo[2,3-b]pyridin-1-yl]ethoxy}phenyl)-2-ethoxypropanoic acid,
3-(4-{2-[6-(cyclopropylcarbonyl)-1H-pyrrolo[2,3-b]pyridin-1-yl]ethoxy}phenyl)-2-(2,2,2-trifluoroethoxy)propanoic acid,
3-(4-{2-[6-(cyclohexylcarbonyl)-1H-pyrrolo[2,3-b]pyridin-1-yl]ethoxy}phenyl)-2-ethoxypropanoic acid,
(2S)-3-(4-f{2-[6-(cyclohexylcarbonyl)-1H-pyrrolo[2,3-b]pyridin-1-yl]ethoxy}phenyl)-2-ethoxypropanoic acid,
3-(4-f{2-[6-(cyclohexylcarbonyl)-1H-pyrrolo[2,3-b]pyridin-1-yl]ethoxy}phenyl)-2-(2,2,2-trifluoroethoxy)propanoic acid,
2(S)-3-(4-{2-[6-(cyclohexylcarbonyl)-1H-pyrrolo[2,3-b]pyridin-1-yl]ethoxy}phenyl)-2-(2,2,2-trifluoroethoxy)propanoic acid,
methyl 3-(4-{2-[6-(cyclopentylcarbonyl)-1H-pyrrolo[2,3-b]pyridin-1-yl]ethoxy}phenyl)-2-ethoxypropanoate,
3-(4-{2-[6-(cyclopentylcarbonyl)-1H-pyrrolo[2,3-b]pyridin-1-yl]ethoxy}phenyl)-2-ethoxypropanoic acid.

43. A pharmaceutical composition comprising as active ingredient at least one compound of claim 30, or a pharmaceutically acceptable addition salt thereof with an acid or a base, alone or in combination with one or more pharmaceutically acceptable excipients.

44. A pharmaceutical composition comprising as active ingredient at least one compound of claim 38, or a pharmaceutically acceptable addition salt thereof with an acid or a base, alone or in combination with one or more pharmaceutically acceptable excipients.

45. A pharmaceutical composition comprising as active ingredient at least one compound of claim 41, or a pharmaceutically acceptable addition salt thereof with an acid or a base, alone or in combination with one or more pharmaceutically acceptable excipients.

46. A pharmaceutical composition comprising as active ingredient at least one compound of claim 42, or a pharmaceutically acceptable addition salt thereof with an acid or a base, alone or in combination with one or more pharmaceutically acceptable excipients.

47. A method of treating a living animal body, including a human, afflicted with a condition selected from hyperglycaemia, dyslipidaemia and, more especially, in the treatment of non-insulin-dependent type II diabetes, insulin resistance, glucose intolerance, disorders associated with syndrome X, coronary artery disease and other cardiovascular diseases, renal disorders, retinopathy, disorders associated with the activation of endothelial cells, psoriasis, polycystic ovary syndrome, dementia, osteoporosis, intestinal inflammatory disorders, myotonic dystrophy, pancreatitis, arteriosclerosis, xanthoma, type I diabetes, obesity, conditions requiring regulation of appetite, anorexia, bulimia, anorexia nervosa, cancer, and conditions requiring an angiogenesis inhibitor, comprising the step of administering to the living animal body, including a human, an amount of a compound of claim 30 which is effective for treatment of the condition.

48. The method of claim 47, wherein the cancer is selected from hormone-dependent cancers, such as breast cancer and colon cancer.

49. A composition comprising a combination of the compound of claim 30 and an antioxidant agent.

50. The composition of claim 49, wherein the compound of claim 30 is (2S)-3-[4-(2-{6-[(methoxyimino)(phenyl)methyl]-1H-pyrrolo[2,3-b]pyridin-1-yl}ethoxy)phenyl]-2-(2,2,2-trifluoroethoxy)propanoic acid, an enantiomer or diastereoisomer thereof or a pharmaceutically acceptable addition salts thereof with an acid or a base.

51. The composition of claim 49, wherein the antioxidant agent is coenzyme Qlo.

52. The composition of claim 49, wherein the antioxidant agent is vitamin E.

53. The composition of claim 49 which is (2S)-3-[4-(2-{6-[(methoxy-imino)(phenyl)methyl]-1H-pyrrolo[2,3-b]pyridin-1-yl}ethoxy)phenyl]-2-(2,2,2-trifluoroethoxy)propanoic acid and coenzyme Q₁₀.

54. The composition of claim 49, further comprising one or more pharmaceutically acceptable excipients.

55. A method of treating a living animal body, including a human, afflicted with obesity, comprising the step of administering to the living animal body, including a human, an amount of a composition of claim 49 which is effective for treatment of obesity.

56. A method of treating a living animal body, including a human, afflicted with overweight characterised by a body mass index greater than 25 and less than 30, comprising the step of administering to the living animal body, including a human, an amount of a composition of claim 49 which is effective for treatment of overweight characterised by a body mass index greater than 25 and less than 30.

57. A method of treating a living animal body, including a human, afflicted with obesity induced by therapeutic treatment, comprising the step of administering to the living animal body, including a human, an amount of a composition of claim 49 which is effective for treatment of obesity induced by therapeutic treatment.

58. A method of treating a living animal body, including a human, afflicted with obesity induced by treatment of type I or type II diabetes, comprising the step of administering to the living animal body, including a human, an amount of a composition of claim 49 which is effective for treatment of obesity induced by treatment of type I or type II diabetes.

59. The method of claim 56, wherein the body mass index greater than 25 and less than 30 is caused by therapeutic treatment.

60. The method of claim 56, wherein the body mass index greater than 25 and less than 30 is caused by therapeutic treatment of type I or type II diabetes.
